(19) United States
(12) Patent Application Publication
(10) Pub. No.: US 2013/0226026 A1
(43) Pub. Date: Aug. 29, 2013

(54) PULMONARY NODULE ACCESS DEVICES AND METHODS OF USING THE SAME

(71) Applicant: Spiration, Inc., (US)

(72) Inventors: David H. Dillard, Grapeview, WA (US); Xavier Gonzalez, Woodinville, WA (US); Peter Hoffman, Seattle, WA (US); Desmond O'Connell, Lake Forest Park, WA (US); Christopher Thralls, Seattle, WA (US)

(73) Assignee: SPIRATION, INC., Redmond, WA (US)

(21) Appl. No.: 13/778,008

(22) Filed: Feb. 26, 2013

Related U.S. Application Data
(60) Provisional application No. 61/604,462, filed on Feb. 28, 2012.

(51) Int. Cl. A61M 25/04; A61B 19/00; A61B 10/02; A61M 25/09; A61M 25/01

(52) U.S. Cl. CPC A61M 25/04; A61M 25/09; A61M 25/0108; A61B 10/02; A61B 19/54
USPC 600/562; 604/174; 604/104; 604/506

(57) ABSTRACT

In certain embodiments, a device is configured to be delivered to the site of a nodule in the lung or other body organ or lumen. The device can be configured to attach directly to the target nodule or to tissue adjacent the target nodule. The device can be configured to provide repeatable access to the site of the target nodule. In some embodiments, the device includes a guide wire that can extend proximal the device. The guide wire can be configured to engage with a catheter or other medical device to provided repeatable and dependable navigation to the site of the target nodule. In some embodiments, the device can include a channel portion through which medical devices and/or instruments can be navigated. In some embodiments, the device can be deployed in the wall of an airway or other body lumen to provide transluminal access to a nodule outside of the lumen.

### RELATED APPLICATIONS

Any and all priority claims identified in the Application Data Sheet, or any correction thereto, are hereby incorporated by reference under 37 CFR 1.57.

### BACKGROUND

1. Technical Field

Embodiments of the disclosed devices generally relate to the field of medical devices, and in particular, to methods, systems, and devices for accessing and/or providing repeatable access to regions in the lung and other internal organs.

2. Description of the Related Art

Treatment or investigation of nodules, lesions, or pathological areas in the lung often requires repeated access to the same region of the lung. In some cases, test to determine whether the nodule is benign or malignant can take days or weeks and can require multiple biopsy samples from the same nodule. Treatment of malignant nodules can require further repeated access to treat the nodule. In cases where a nodule is located in the peripheral regions of the lung, navigation and access can be challenging because the small diameters of the airways in the peripheral regions of the lung do not admit to visual navigation. There is therefore a need for a device and method to safely, quickly, and consistently access the site of a nodule on a repeatable basis.

### SUMMARY

In some embodiments, a device for providing repeatable access to a nodule or other area of interest within the body can include a fixation portion. The fixation portion can be configured to attach to tissue in the body. The fixation portion can have a proximal end and a distal end. In some embodiments, the fixation portion can include one or more anchors on the proximal end and/or the distal end of the fixation portion. In some embodiments, the one or more anchors can be fixed in a substantially constant position relative to the target nodule or other area of interest. In some embodiments, the device can include a guide portion. The guide portion can be configured engage with an instrument. The instrument can be configured to navigate along the guide portion of the device to the fixation portion of the device. In some embodiments, the device can be configured to remain deployed for a short term (e.g. for one procedure). In some embodiments, the device can be configured to remain deployed for an extended and/or permanent period of time.

Various example embodiments of the disclosure can be described in view of the following clauses:

Clause 1: a device for providing access to a nodule in a lung or other body organ or lumen, the device comprising: a channel portion, the channel portion having a proximal end and a distal end, wherein the channel portion defines a lumen, the lumen extending from the proximal end of the channel portion to the distal end of the channel portion; and at least one anchor member, the anchor member configured to inhibit rotational, proximal, and distal motion of the channel portion with respect to the nodule upon deployment of the device in a body lumen.

Clause 2: The device of Clause 1, wherein the channel potion further comprises at least one radiopaque marker.

Clause 3: The device of any of Clauses 1 or 2, wherein the device further comprises a guide member.

Clause 4: The device of Clause 3, wherein the guide member comprises a guide wire.

Clause 5: The device of any of Clauses 3 or 4, wherein the guide member comprises a guide tube.

Clause 6: The device of any of Clauses 3-5, wherein the guide member comprises at least one radiopaque marker.

Clause 7: The device of any of Clauses 1-6, wherein the channel portion further comprises one or more cut portion, the one or more cut portions configured to increase the flexibility of the channel portion.

Clause 8: The device of Clause 7, wherein channel portion further comprises a heat shrink.

Clause 9: The device of any of Clauses 1-8, wherein the channel portion is further configured to deploy transluminally in the wall of an airway or other body lumen.

Clause 10: The device of any of Clauses 1-9, wherein the channel portion is further configured to transition between a compressed state within a working channel of a catheter or other delivery device and an expanded state upon deployment in an airway or other body lumen.

Clause 11: The device of any of Clauses 1-10, wherein the channel portion further comprises a port between the proximal end of the channel portion and the distal end of the channel portion.

Clause 12: The device of any of Clauses 1-11, wherein the at least one anchor member comprises a piercing portion configured to pierce tissue at or near the nodule, and wherein the at least one anchor member comprises a pad portion configured to limit a depth to which the piercing portion pierces the tissue.

Clause 13: The device of any of Clauses 1-12, wherein the channel portion comprises a first anchor coupled with the proximal end of the channel portion and extending proximally from the proximal end of the channel portion when the device is deployed.

Clause 14: The device of any of Clauses 1-13, wherein the channel portion comprises a first anchor coupled with the distal end of the channel portion and extending distally from the distal end of the channel portion when the device is deployed.

Clause 15: The device of Clause 1, wherein the device further comprises a directional member coupled with the distal end of the channel member, the directional member configured to direct the distal end of the channel member toward a wall of an airway or other body lumen upon deployment of the device in an airway or other body lumen.

Clause 16: The device of Clause 15, wherein the distal end of the channel member has an echogenically unique portion configured to identify the orientation of the directional member.

Clause 17: The device of Clause 16, wherein the channel member can be rotated prior to deployment to rotationally align the echogenically unique portion with respect to the nodule.

Clause 18: The device of any of Clauses 15-17, wherein the channel member can be rotated prior to deployment to rotationally align the directional member with respect to the nodule.

Clause 19: The device of Clause 15-18, wherein the anchor member engages the lumen wall upon deployment of the device, engagement of the anchor member with the lumen wall fixing the rotational alignment of the directional member with the nodule.

Clause 20: The device of any of Clauses 15-19, wherein the directional member comprises one or more projections.

Clause 21: A method of deploying and using a fiducial device for repeatable access to a nodule in a lung or other body organ, the method comprising: locating a target nodule in the body; compressing the fiducial device within the working channel of a catheter or other delivery device, the fiducial device comprising a fixation portion and a guide portion; navigating the catheter or other delivery device to the site of the target nodule; removing the fiducial device from the working channel of the catheter or other delivery device; attaching the delivery device to tissue proximate the target nodule; removing the catheter or other delivery device from the site of the target nodule; engaging a second catheter or other delivery device with the guide portion of the fiducial device, the second catheter of other delivery device including a treatment or diagnosis instrument; navigating the second catheter or other delivery device along the guide portion of the fiducial device to the site of the target nodule; treating or collecting a sample from the target nodule; and withdrawing the second catheter or other delivery device along the guide portion of the fiducial device.

Clause 22: A device for providing access to a nodule in a lung or other body organ or lumen, the device comprising: a fixation portion having a proximal end and a distal end, the fixation portion comprising one or more anchor portions, the fixation portion configured to be attached to tissue in substantially constant proximity to the nodule; and a guide portion having a proximal end and a distal end, the guide portion configured to removably engage with a medical instrument, the guide portion further configured to guide the navigation of the medical instrument to the fixation portion.

Clause 23: The device of Clause 22, wherein the one or more anchor portions are configured to removably attach to tissue.

Clause 24: The device of any of Clauses 22-23, wherein the fixation portion is attached directly to the nodule.

Clause 25: The device of any of Clauses 22-24, wherein the fixation portion is attached proximal to the nodule.

Clause 26: The device of any of Clauses 22-25, wherein the guide portion comprises a guide wire.

Clause 27: The device of any of Clauses 22-26, wherein the guide portion comprises a guide channel.

Clause 28: The device of any of Clauses 22-27, wherein the guide portion further comprises one or more anchors on the proximal end of the guide portion.

Clause 29: The device of any of Clauses 22-28, wherein the fixation portion further comprises one or more radiopaque markers.

Clause 30: The device of any of Clauses 22-29, wherein the guide portion further comprises one or more radiopaque markers.

Clause 31: The device of any of Clauses 22-30, wherein the device is configured to transition between a compressed state within a working channel of a catheter or other delivery device and an expanded state within an airway or other body lumen.

Clause 32: The device of any of Clauses 22-31, wherein the fixation portion is configured to attach to an airway wall.

### BRIEF DESCRIPTION OF THE DRAWINGS

FIG. 1 is a schematic cross-sectional view of an airway or body lumen and three types of nodules.

FIG. 2 is a view of wire-based nodule access device.

FIG. 3 is a view of the device of FIG. 2 deployed within an airway or body lumen.

FIG. 4A is a view of a rounded end cap that can be used on a proximal end of the device of FIG. 2.

FIG. 4B is a view of a bristled end that can be used on the proximal end of the device of FIG. 2.

FIG. 4C is a view of a pigtail end that can be used on the proximal end of the device of FIG. 2.

FIG. 4D is a view of a loop end that can be used on the proximal end of the device of FIG. 2.

FIG. 5 is a side view of a delivery device showing an attached side car configuration mated over the device of FIG. 2.

FIG. 6 is a side view of a multi-lumen delivery device with a lumen mated over the device of FIG. 2.

FIG. 7A is a perspective view of a delivery device with a wrapped lasso portion mated over the device of FIG. 2.

FIG. 7B is a side view of a delivery device with an end cap and a lasso portion mated over the device of FIG. 2.

FIG. 7C is a top view of the delivery device of FIG. 7B.

FIG. 8 is a view of a nodule access device engaged with a multi-lumen delivery device within an airway or other body lumen.

FIG. 9 is an enlarged view of the nodule access device and multi-lumen delivery device of FIG. 8 where a needle is inserted into a target nodule.

FIG. 10A is a schematic cross-section view of a nodule access device delivery apparatus including a Tuohy connector, a coil channel, and a push rod.

FIG. 10B is a view of the delivery apparatus of FIG. 10A with a partially-deployed nodule access device.

FIG. 11A is a simplified cross-section view of a delivery apparatus and a wall anchor.

FIG. 11B is a simplified cross-section view of the interior of a channel in which the wall anchor of FIG. 11A is compressed.

FIG. 12 is a view of an embodiment of an access port and braided channel deployed in an airway or other body lumen.

FIG. 13 is a view of an embodiment of an access port and a coil channel deployed in an airway or other body lumen.

FIG. 14 is a view of an embodiment of an access port and a coil channel deployed in an airway or other body lumen.

FIG. 15A is a perspective view of an access channel.

FIG. 15B is a perspective view of the access channel of FIG. 15A deployed within an airway or other body lumen.

FIG. 15C is a perspective view of an embodiment of an access channel with an attached guidewire deployed in an airway or other body lumen.

FIG. 15D is a perspective view of an embodiment of an access channel with an attached guidewire having distance markers.

FIG. 16 is a perspective view of an embodiment of an access channel deployed within an airway or other body lumen.

FIG. 17A is a top view of a flat coil access channel with a distal spigot.

FIG. 17B is a side view of an embodiment of a flat coil access channel with a distal spigot and a plurality of anchors.

FIG. 17C is a side view of an embodiment of a flat coil access channel with varied pitch portions.

FIG. 17D is a perspective view of an embodiment of a flat coil access channel with a pre-set bend.

FIG. 18 is a perspective view of a tunneling device within an airway or other body lumen.

FIG. 19A is a view of a port deployment apparatus with a distal cone member and a push coil.

FIG. 19B is a view of the port deployment apparatus of FIG. 19A where the anchors of the port are deployed.

FIG. 20A is a side view of a transluminal access port compressed within the working channel of a delivery device.

FIG. 20B is a side view of the transluminal access port of FIG. 20A in a deployed state.

FIG. 21A is a side view of the transluminal access port of FIG. 20A shortly after deployment of the access port in an airway wall.

FIG. 21B is a view of the transluminal access port of FIG. 21A that has expanded over time after deployment in an airway wall.

FIGS. 22A-22D are perspective views of a delivery device with a side working channel during the delivery of a transluminal access port into an airway wall.

FIG. 23A is a perspective view of a coil embodiment of a transluminal access port.

FIG. 23B is a perspective view of a coil anchor embodiment of a transluminal access port.

FIG. 23C is a perspective view of an end anchor embodiment of a transluminal access port.

FIG. 23D is a side view of a cut transluminal access port with wall-engaging fingers.

FIG. 23E is a side view of the cut transluminal access port of FIG. 23D after installation in an airway wall.

FIG. 23F is a side view of a cut transluminal access port with wall-engaging web end portions.

FIG. 24 is a view of the distal end of a device with hook anchors.

FIG. 25 is a view of a channel access device with directional members on the distal end and a basket portion on the proximal end.

FIG. 26 is a view of the proximal end of a channel access device having outwardly-directed flat panel portions.

### DETAILED DESCRIPTION

Devices and methods for providing repeatable access to nodules, lesions, or pathological areas in the lung or other bodily organ will now be described with reference to the accompanying figures of one or more embodiments. The terminology used in the description presented herein is not intended to be interpreted in any limited or restrictive manner. Rather, the terminology is simply being utilized in conjunction with a detailed description of embodiments of the devices and methods. For example, the term “nodule” can refer to lesions, tumors, or other pathologies within the body, independent of size and shape. Such nodules could include cancer tumors that need diagnosis and/or treatment, tuberculosis lesions that need diagnosis and/or treatment, loculated infections that need to be drained and/or treated with antibiotics, and/or bullae that need to be decompressed and/or otherwise treated.

Some or all of the embodiments herein disclosed can be utilized to provide repeatable access to a nodule or other site of interest for sampling, taking biopsies, or otherwise diagnosing the site of interest. Furthermore, some or all of the embodiments can be used to provide repeatable access to a site of interest for the purpose of providing treatment to the site of interest. For example, the embodiments herein may be utilized to provide repeatable access to the site(s) of interest for the purpose of administering medicants (e.g., chemotherapy) and/or administering energy and/or therapeutic seeds to the site of interest. Tools for draining infections (e.g., loculated infections) and/or bullae, providing antibiotics, and/or for introducing sealants to a site of interest can be used with some or all of the embodiments described herein.

Furthermore, embodiments may comprise several novel features, no single one of which is solely responsible for its desirable attributes or is believed to be essential to practicing the inventions herein described. Although some embodiments described herein refer to deploying an access device into an airway, this disclosure is not so limited, and deployment could be made, for example but without limitation, into other vessels, passages, and body cavities in humans and animals. Additionally, the embodiments described herein could be configured to be removable or permanent, depending on the purpose behind deploying the given embodiment in a given procedure. In some embodiments, the device can comprise a plurality of components which can be configured to connect to and/or disconnect from each other (e.g. proximal, central, and distal components). In such embodiments, the device can be configured to be completely (e.g. all components) removable and/or partially removable (e.g. some components). Some embodiments of the device can be completely permanent (e.g. call components permanently deployed) and/or partially permanent (e.g. some components removable). Some of the embodiments described herein can be used in conjunction with a number of treatment and/or diagnosis instruments (e.g. cytology brushes, RF probes, ultrasound probes, biopsy forceps, TBNA needles, etc.). Each of the embodiments described herein could comprise radiopaque markings or other visualization aids (e.g. markings compatible with x-ray, CT and/or bronchoscopic visualization) to assist a care provider in navigating, deploying, and/or locating the device. Some of the embodiments described herein can include laser cut patterns, side passageways, or other features detectable by an ultrasound probe or other visualization device.

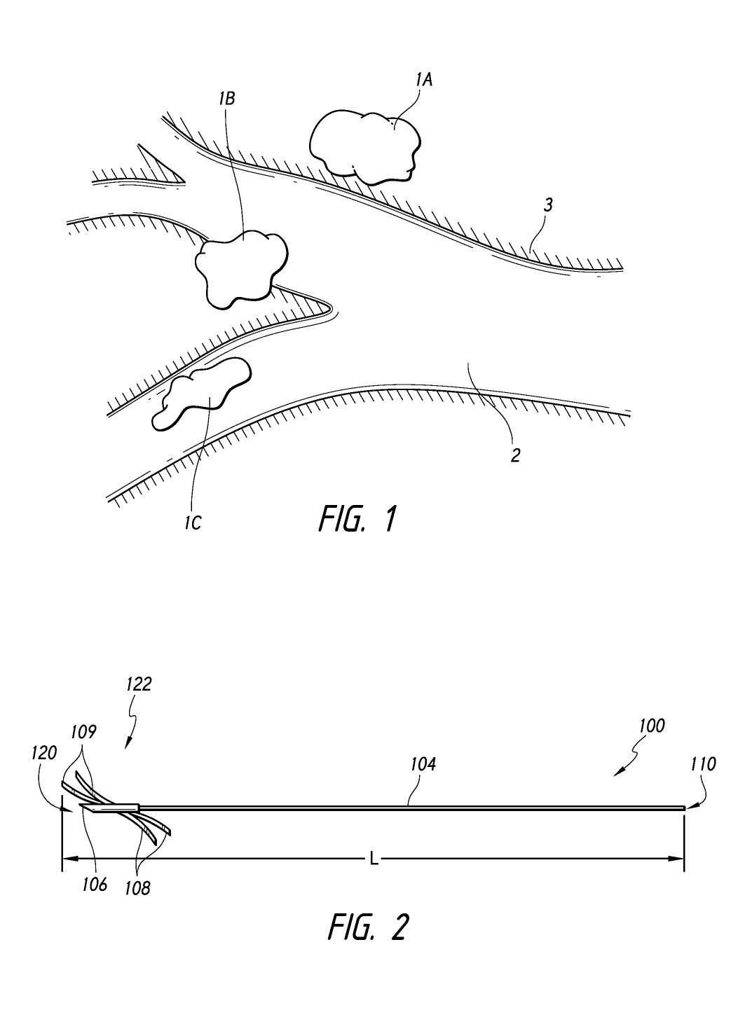

FIG. 1 illustrates an airway 2 having a number of nodules therein. In general, nodules can be grouped into three or more types. For example, nodules located outside of an airway passage are generally referred to as extrinsic nodules 1A. Nodules that span an airway wall 3 generally are referred to as mixed nodules 1B. Nodules that are located within an airway 2 are generally referred to as intrinsic nodules 1C. Each type of nodule presents its own challenges for access and treatment. Desirably, consistent and repeated access to any particular site proximate a nodule can be accomplished using one or more of the devices described herein. Advantageously, one or more of the devices described herein can be directly anchored proximate or at a region containing a site of interest such that the device will move with the site. For example, by anchoring to the tissue, airway, or other portion of the body that is adjacent to or that contains the region of interest, as the patient or recipient breaths or has other anatomical movement, one or more of the devices described herein will move with the region of interest. This provides distinct advantages over catheters, lumens and the like that provide a frame of reference to a location external to the body or the location of interest, for example. Such catheters, lumens and the like have a distal end that does not move with movement of the region of interest. For example, if the patient is breathing, relative movement between the lung tissue and the end of a catheter, bronchoscope or the like will occur with each breath. In some configurations, the devices or at least some portion of the devices described herein can function as fiducial markers. In some arrangements, more than two of the devices or more than two portions of one or more devices can be used to define a plane. Thus, the devices, or portions of the devices, can be used to locate nodules, or other sites of interest, visually or through other suitable techniques.

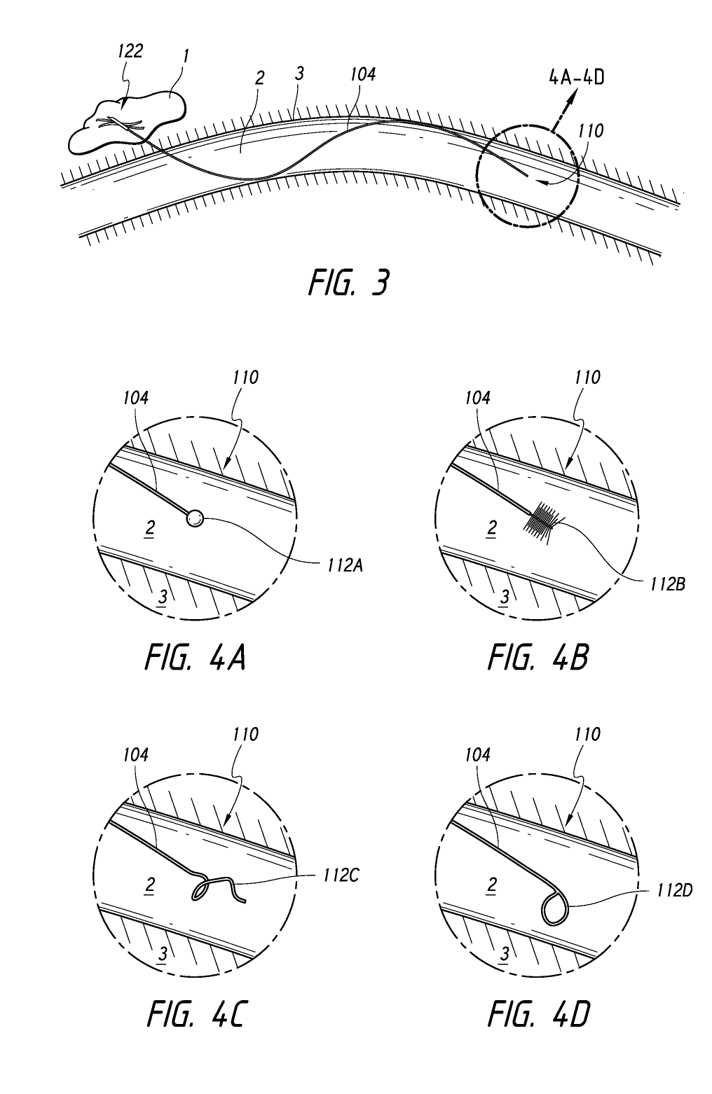

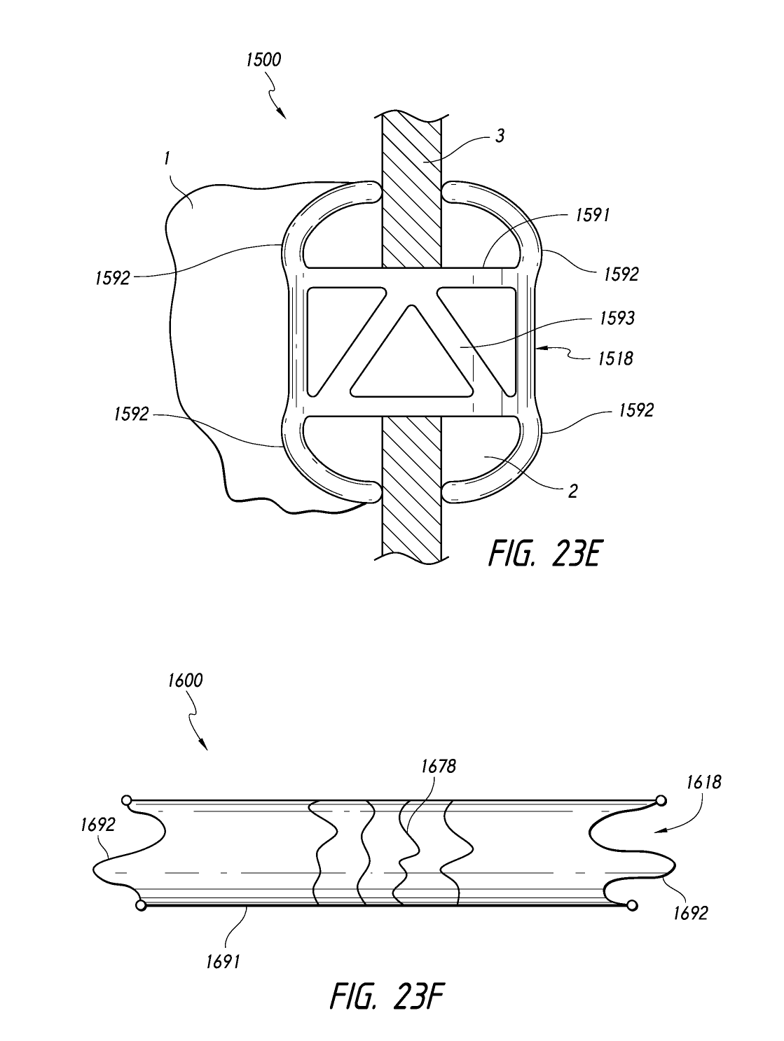

FIG. 2 illustrates a device for providing repeated access to a site of interest. In some embodiments, a wire-based device 100 can have a distal end 120 and a proximal end 110. The wire-based device 100 can include a fixation portion 122 on the distal end 120. The fixation portion 122 can have a piercing member 106. In some embodiments, the wire-based device 100 includes one or more anchors 108,109. The device 100 can have one or more distal anchors 109 attached to the distal end of the fixation portion 122 of the device 100. In some embodiments, the wire-based device 100 can include one or more proximal anchors 108. The one or more proximal anchors 108 can be connected to the proximal end of the fixation portion 122. In some configurations, the proximal anchors 108 resist proximal movement while the distal anchors resist distal movement. In other configurations, the proximal anchors can resist distal movement while the distal anchors resist proximal movement. Such a configuration is shown, for example, in FIG. 2, for example but without limitation. In some embodiments, such as the embodiment illustrated in FIG. 24, the fixation portion 122 can include one or more grappling hooks 108A that can be used to secure the device 100 in place within the lung or other body organ or lumen. Other configurations also have been illustrated.

In some embodiments, the anchors 108,109 and/or hooks 108A can be configured to transition between a compressed, relaxed or nondeployed state (e.g., to fit within the working channel of a catheter or other delivery device) and an expanded state in response to mechanical, electrical, thermal, and/or other inputs. In some embodiments one or more of the anchors 108,109 and/or hooks 108A can be constructed of a shape memory material such that the one or more of the anchors 108,109 and/or hooks 108A can remain in a compressed, relaxed or nondeployed configuration at temperatures below body temperature (e.g. 98.6° F. for a human) and transition to an expanded state at temperatures at or above body temperature. In some embodiments, the anchors 108,109 and/or hooks 108A can be formed from a bimetallic strip that can bend outward into the body tissue in response to body heat and can return to a straight, compressed configuration in response to cooling of the bimetallic strip. In some embodiments, the anchors 108,109 and/or hooks 108A can comprise hollow channels into which a bent wire could be inserted. As the bent wire is pushed toward the distal end of the hollow anchors 108,109 and/or hooks 108A, the anchors 108,109 and/or hooks 108A could bend to match the shape of the bent wire and engage with the surrounding body tissue. In some embodiments, the anchors themselves can be formed by one or more bent wire that bends as it moves outward from a sheath. Other configurations are possible.

With reference again to FIG. 2, in some embodiments, the wire-based device 100 includes a guide tail or guide wire 104. The guide wire 104 can extend from the proximal side of the piercing member 106 and/or the anchors 108,109. The wire-based device 100 can have an overall length L. In some embodiments, the length L is greater than about 2 cm and/or less than about 15 cm. In some embodiments, the length L is approximately 10 cm. The length of the device 100 can be shortened prior to or following installation within a passage. In some configurations, the length of the device 100 is sufficiently long such that, when a distal end is anchored, the proximal end can be located visually with a bronchoscope, an endoscope or the like.

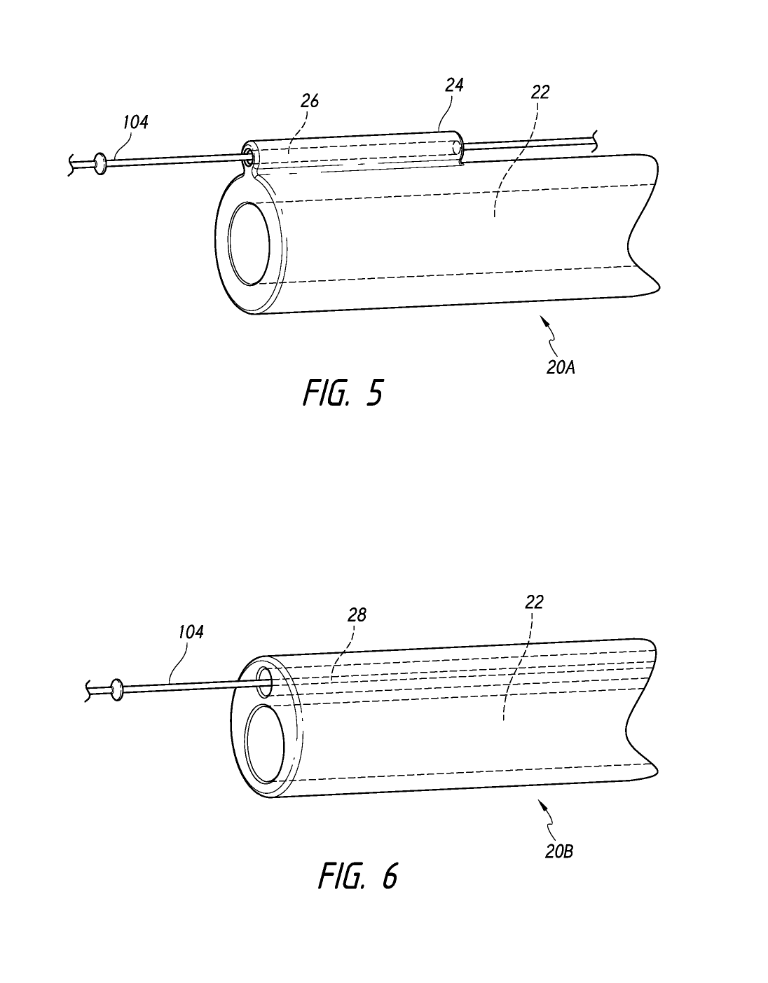

As illustrated in FIG. 3, the wire-based device 100 can be used to access a peripheral nodule 1. The fixation portion 122 can be inserted into a nodule 1 directly. In some configurations, the fixation portion 122 can be inserted into a sidewall 3 of an airway 2 at a location proximate or proximal to the nodule 1. In some configurations, the fixation portion 122 can be inserted into the sidewall 3 of an airway 2 at a location adjacent to or distal to the nodule 1. In some embodiments, the fixation portion 122 is engaged with tissue at or near the target nodule 1 such that the relative position between the fixation portion 122 and the target nodule 1 remains substantially constant as the patient breathes and otherwise moves his or her body. This same substantially constant positioning between the fixation portion 122 and the nodule 1 can be obtained with any of the embodiments of access devices described herein. The guide wire 104 can extend proximally into an airway 2 from the proximal end of the fixation portion 122. In some situations, the guide wire 104 can pass proximally from the fixation portion 122 through one or more generations of the bronchial tree. A guide sheath or other structure or implement can be used to lift the guide wire 104 from a location proximate an airway wall. With the proximal end of the guide wire 104 raised, a guide sheath can slide over at least a portion of the guide wire 104 to allow repeated navigation to a site. In some configurations, a treatment and/or diagnosis instrument can be passed along either the guide wire 104 or an attached guide sheath to a fixed location with respect to the target nodule 1 or other site of interest.

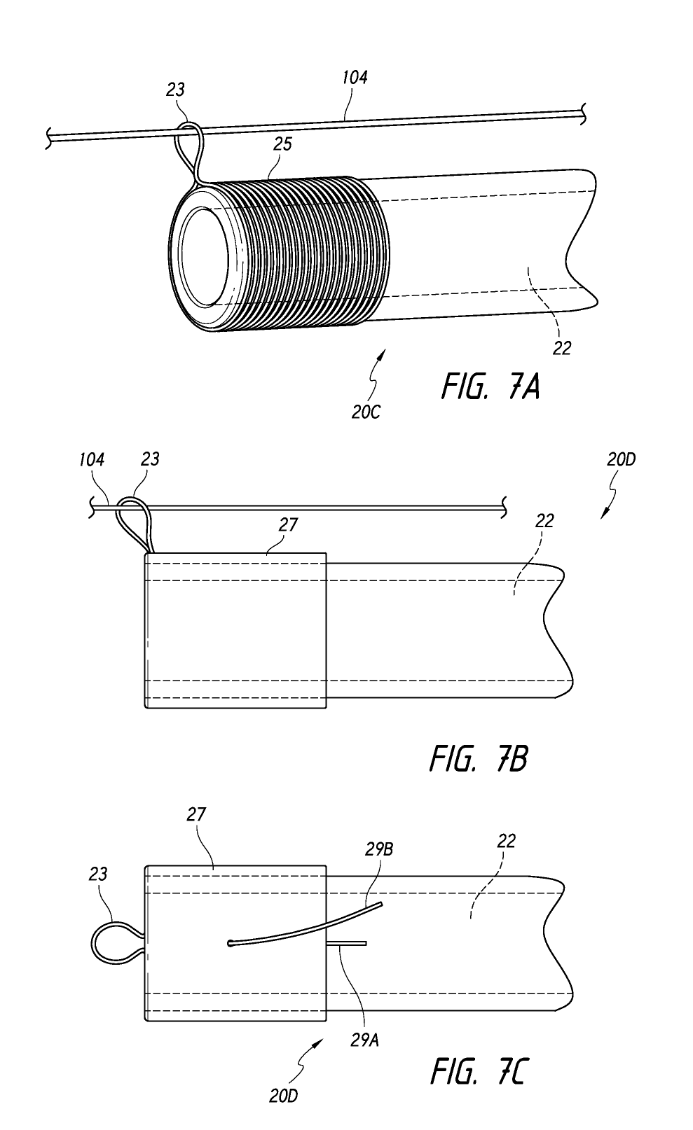

FIGS. 4A-4D illustrate embodiments of proximal structures that can help to reduce the likelihood that the proximal end 110 of the guide wire 104 will puncture or adhere to the sidewall 3 of an airway 2 and to facilitate the pick-up of a guide sheath or the like. For example, FIG. 4A illustrates a guide wire 104 with a rounded member 112A. The rounded member 112A can have a spherical, hemispherical, disc-shaped, oval or any other similar shape. While the illustrated rounded member 112A is shown at the extreme proximal end, the rounded member 112A can be positioned distal (e.g., slight distal) of the extreme proximal end in some embodiments. In some embodiments, the proximal end 110 of the guide wire 104 can have a bristled end 112B, as illustrated in FIG. 4B. In some embodiments, the proximal end 110 of the guide wire 104 can have a pigtail configuration 112C, and in some embodiments, the proximal end 110 of the guide wire 104 can have a loop configuration 112D, as illustrated in FIGS. 4C and 4D, respectively. In some embodiments, the proximal end 110 of the guide wire 104 can have a beetle design that rolls over and hooks onto the guide wire 104. Each of these embodiments, as well as any additional embodiments of the proximal end 110 of the guide wire 104 can help reduce the likelihood that the proximal end 110 of the guide wire 104 will adhere to or puncture the airway wall 3. Some of the embodiments listed above can be located distal to the proximal end 110 of the guide wire 104. Moreover, the structures preferably raise the proximal end 110 of the guide wire 104 such that the proximal end 110 is easier to engage with another structure or component, like a guide sheath, for example but without limitation.

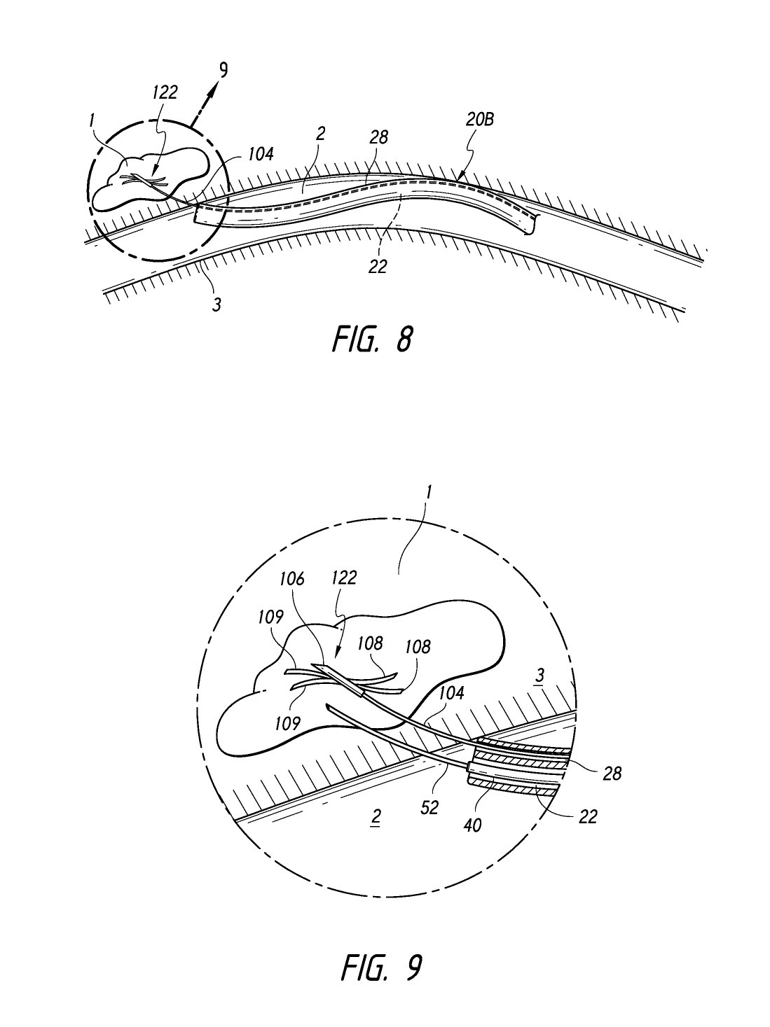

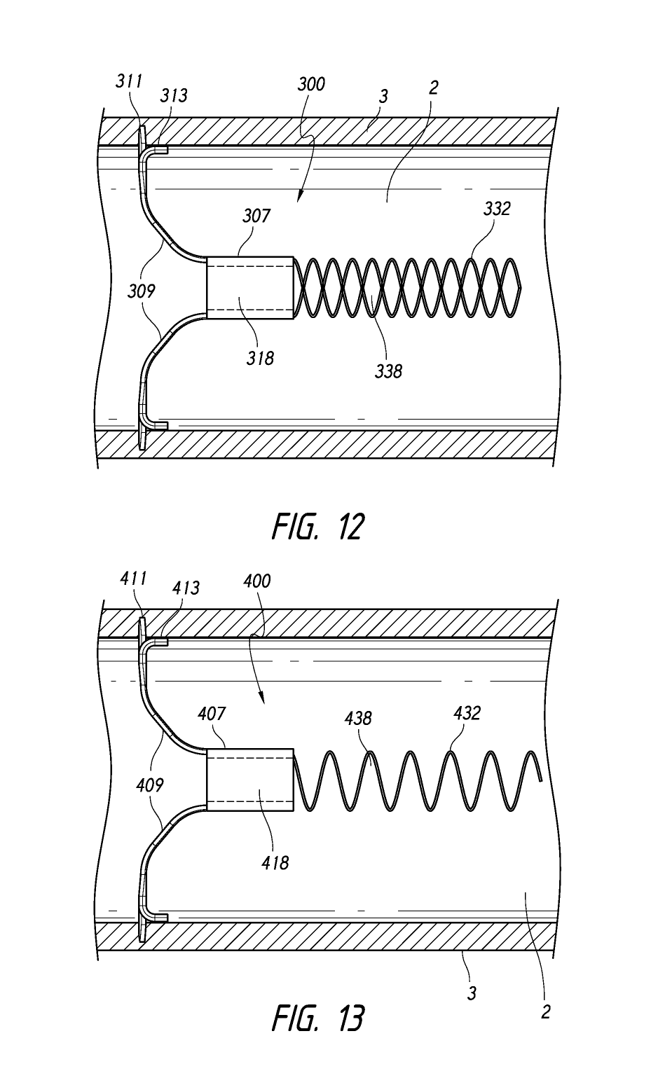

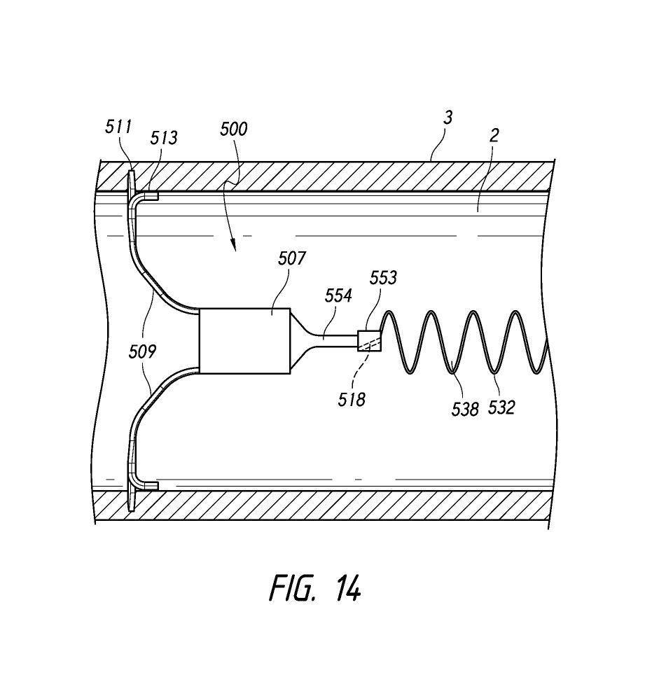

In some situations, repeated access to the nodule 1 or other desired location can be accomplished by guiding a catheter 20 (see, e.g., FIGS. 8 and 9) or other medical device along the guide wire 104 of the access device. For example, a catheter 20A could include a guide structure such as a side car 24 at or near the distal end of the catheter 20A, as illustrated in FIG. 5. The side car 24 could include a guide wire channel 26 that can be configured to coaxially engage with the guide wire 104 of the access device. In some embodiments, upon engagement between the guide wire channel 26 and guide wire 104, the catheter 20A can be directed along the guide wire 104 to the site of the fixation portion of the access device near the site of a nodule 1 or other desired location. The catheter 20A can include a working channel 22 through which instruments (e.g. biopsy forceps, cytology brushes, RF probes, TBNA needles, ultrasound probes, mini-probes (ultrasonic probes), EndoTherapy devices, etc.) can travel to the site of the target nodule 1 or other desired location.

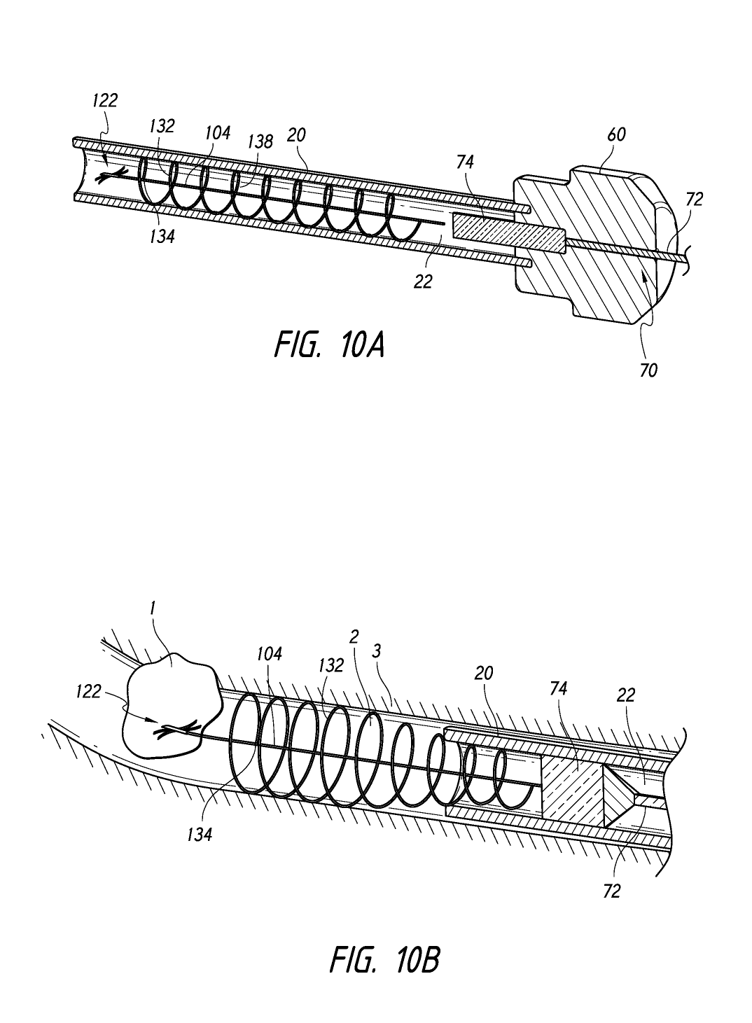

In some embodiments, a catheter 20B could have two or more lumens 22,28, as illustrated in FIG. 6. The catheter 20B could have a guide structure comprising a guide wire lumen 28 that can be configured to coaxially or otherwise engage with the guide wire 104 of an access device. In some embodiments, engagement between the guide wire 104 and the guide wire lumen 28 can allow the catheter 20B to move along the guide wire 104 to the site of the target nodule 1 or other desired location. The catheter 20B can include a working channel 22 through which instruments can travel to the site of the target nodule or other desired location.

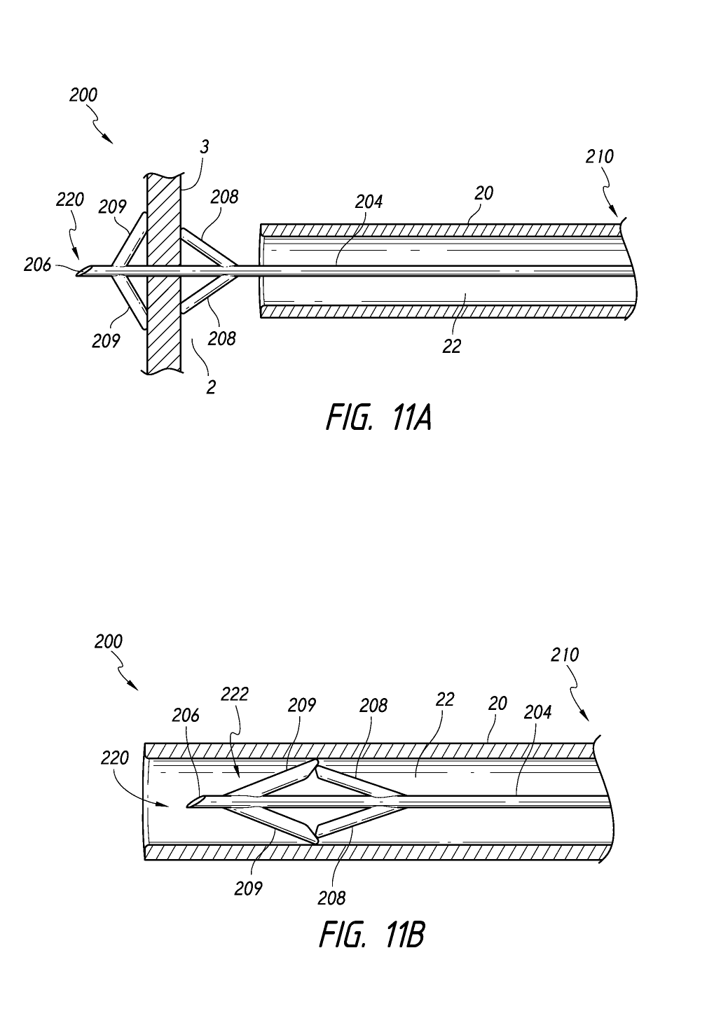

Some catheters 20C can include guide structure comprising a lasso 23. The lasso 23 can be configured to engage with a guide wire 104 of an access device. The lasso 23 can be constructed of nitinol wire or some other resilient or flexible material, for example but without limitation. In some embodiments, the lasso 23 can be formed by wrapping wire around the distal end of a catheter 20C and leaving a loop of wire free to form the lasso, as illustrated in FIG. 7A. In some embodiments, a catheter 20D can include a distal cap 27, as illustrated in FIGS. 7B-7C. In some embodiments, the lasso 23 can have a first end 29A and a second end 29B. The first end 29A can be secured between the body of the catheter 20D and the distal cap 27. The second end 29B of the wire can extend from inside of the distal cap 27 to outside of the end cap 27. In some embodiments, the second end 29B of the lasso 23 can be pulled to tighten the lasso 23. In some embodiments, the lasso 23 can be constructed of shape memory material that changes shape in reaction to heat or cold. For example, the lasso 23 could be configured to fit within the working channel 22 of the catheter 20D when the catheter is subject to room temperature and the lasso 23 could be configured to “flip” up and out of the working channel 22 when subject to higher temperatures within a patient's body. The lasso 23 could be configured to change shape in a variety of additional configurations.

In some embodiments, the guide wire 104 includes a stop structure (e.g., a tab, a disc, a bulbous structure, etc.) configured to have an effective diameter that is similar to or larger than a diameter of the guide structure of the catheter 20. In some such embodiments, engagement between the stop structure and the guide structure can provide tactile feedback (e.g., a stop, a click, an increase in friction between the catheter and the guide wire) to the user of the catheter 20. The stop structure can be positioned at or near the distal end of the access device to provide a reference point for the location of the catheter 20 with respect to the distal end of the access device.

FIG. 8 illustrates an embodiment of a catheter 20B with a guide wire lumen 28 and a working channel 22. The guide wire lumen 28 is engaged with the guide wire 104. The distal end of the catheter 20B is advanced along the guide wire 104 to the site of the fixation portion 122 of the access device. As illustrated in FIG. 9, an instrument sheath 40 can be inserted through the working channel 22 of the catheter 20B and maneuvered to the distal end of the catheter 20B. A medical instrument 52 (e.g., a TBNA needle as illustrated) can be housed within the protective sheath 40, which can be a portion of the medical instrument 52. In some embodiments, the medical instrument 53 can be a lung biopsy needle, as described in Provisional Application Ser. No. 61/604,457, filed Feb. 28, 2012, titled “LUNG BIOPSY NEEDLE” (Atty. Docket SPIRTN.084PR), and the application is hereby incorporated by reference in its entirety. Further examples of lung biopsy needles for use as the medical instrument 53 are described in U.S. patent application Ser. No. ______, filed Feb. 26, 2013, titled “LUNG BIOPSY NEEDLE” (Atty. Docket SPIRTN.084A), published as U.S. Patent Publication No. ______, and the publication is hereby incorporated by reference in its entirety. The medical instrument 52 can access the target nodule 1 for treatment and/or diagnosis. Upon completion of the diagnosis and/or treatment of the nodule 1, the catheter 20B can be withdrawn along the guide wire 104 until the guide wire lumen 28 disengages from the guide wire 104. The access device and guide wire 104 can remain in the airway(s) after the catheter 20B is withdrawn. Additional catheters 20 or other devices can subsequently access the guide wire 104 and travel to the site of the target nodule 1 for additional treatment and/or diagnosis of the nodule 1, for example but without limitation.

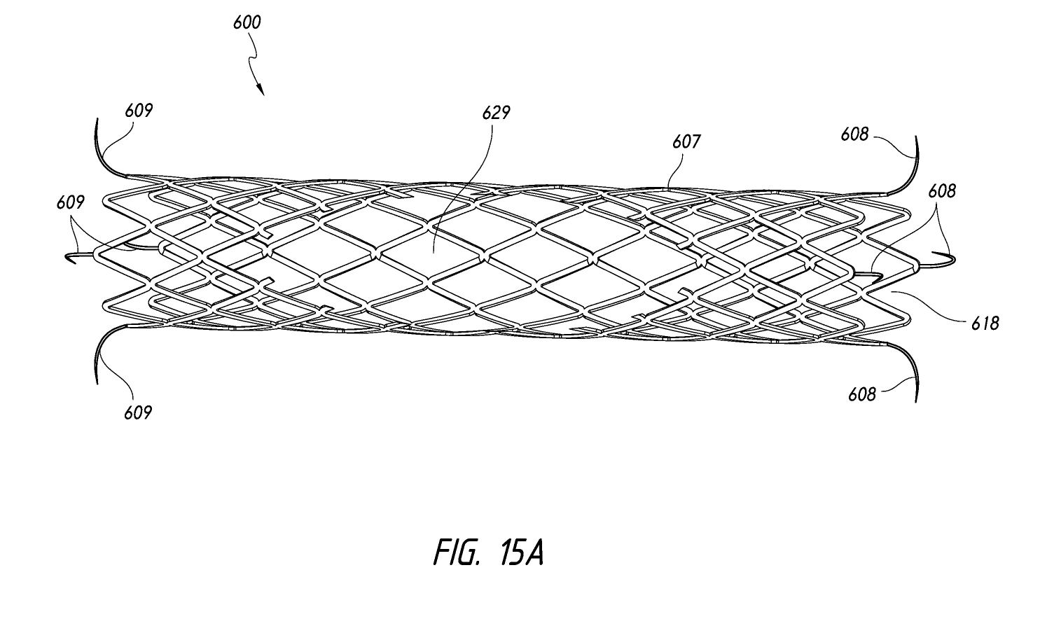

FIG. 10A illustrates an embodiment of a deployment system for an access device. The deployment system can include a guide sheath or catheter 20. In some embodiments, the deployment system includes a push rod 70. The push rod 70 can have a push rod tip 74 and a rod portion 72. The push rod 70 and guide sheath 20 can be concentrically coupled by a Tuohy connector 60, for example but without limitation. In some embodiments, the Tuohy connector 60 can be used to hold the push rod 70 and the guide sheath 20 in a fixed position relative to each other (e.g., the Tuohy can prevent the guide sheath 20 from moving in the proximal and/or distal directions with respect to the push rod 70). In some configurations, the access device can include a coil portion 132. The coil portion 132 can define a coil channel 138. The distal end of the coil portion 132 can be connected to the guide wire 104 of the access device at a connection point 134 by crimping of other similar means. The coil portion 132 and/or fixation portion 122 of the access device can be configured to compress within the guide sheath 20.

To deploy the access device at the site of a nodule 1, the distal end of the guide sheath 20 can be advanced to the site of the nodule 1. The guide sheath 20 can be navigated using ultrasound, fluoroscopy, camera guidance, or any other suitable navigation arrangement (e.g., systems available from Super Dimension, Cybernet Systems' Bf NAVI system, Broncus' LungPoint system and Veran's system). After the distal end of the guide sheath 20 is positioned in the desired location, the Tuohy connector 60 can be loosened. The push rod 70 then can be used to push the access device in the distal direction and allow the fixation portion 122 of the access device to penetrate the nodule 1 and/or the nearby airway wall 3. The guide sheath 20 then can be pulled in the proximal direction to unsheathe the access device and deploy the access device in the airway 2. Preferably, the push rod 70 is held static during unsheathing.

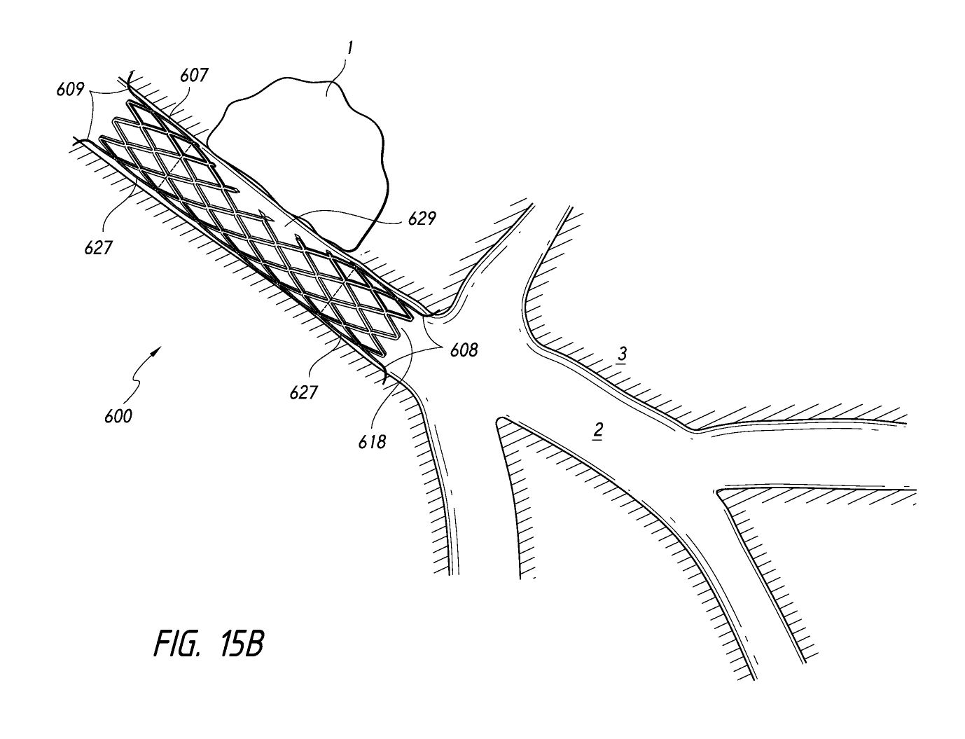

FIGS. 11A and 11B illustrate an embodiment of a wall anchor 200 that can be used to provide repeated access to a specific location within the lung. The wall anchor 200 can have a proximal end 210 and a distal end 220. The wall anchor 200 can include a fixation portion 222 on or near the distal end 220 of the wall anchor 200. In some embodiments, the wall anchor 200 includes one or more distal anchors 209. In some embodiments, the wall anchor 200 includes one or more proximal anchors 208. The fixation portion 222 of the wall anchor 200 can include a piercing member 206. The piercing member 206 can be configured to penetrate airway walls 3 or other bodily tissue. In some embodiments, the wall anchor 200 can include a guide wire or tube 204 extending from the proximal end of the fixation portion 222. In some arrangements, a conical end can be used to define both the piercing member 206 and the distal anchors 209.

The wall anchor 200 can be configured to be compressed into the working channel of a catheter 20 or other deployment apparatus. As illustrated, the anchors 208,209 can be configured such that the distal anchors 209 overlap the proximal anchors 208 when in a compressed configuration. The anchors 208,209 can be constructed of a resilient material such that the anchors 208,209 are biased to the open position (as illustrated in FIG. 11A). In some configurations, the wall anchor 200 can be deployed by pushing the piercing member 206 through an airway wall 3. The fixation portion 222 can be advanced through the wall 3 until the distal anchors 209 expand to an open position. The wall anchor 200 can then be pulled back through the airway wall 3 in the proximal direction until the proximal anchors 208 expand to an open position. The catheter 20 can then be withdrawn, leaving the wall anchor in place in the wall 3 of the airway 2. Subsequent repeatable and quick access to the site of the deployed fixation portion 222 can be achieved by guiding a catheter 20 or other instrument along the guide tube 204 of the wall anchor 200 in any of the manners discussed above.

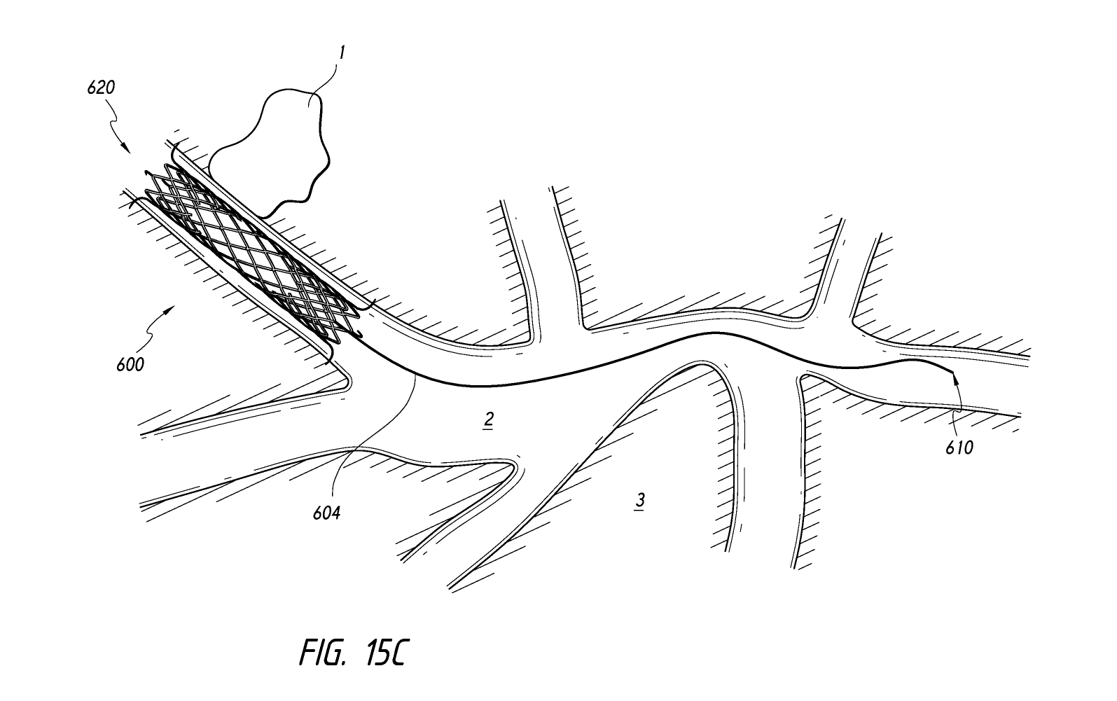

In addition to or alternative to guide wire devices, channel and/or port devices can be used to provide repeatable access to a lung nodule. FIG. 12 illustrates an embodiment of an access port 300 deployed within an airway 2 or other body lumen. The access port 300 can include a port body portion 307. The port body portion 307 can comprise a port channel 318 extending from the proximal end of the port body portion 307 to the distal end of the port body portion 307. In some embodiments, the access port 300 can include a braided wire portion 332 attached to and extending proximally from the proximal end of the port body portion 307. The braided wire portion 332 can include a braided wire channel 338 extending from the proximal end of the braided wire portion 332 to the distal end of the braided wire portion 332. In some embodiments, the braided wire channel 338 can be constructed of a wire made of stainless steel, nitinol, or other suitable material. In some embodiments, the braided wire channel 338 can include an inner liner formed of PTFE, for example but without limitation. In some embodiments, the braided wire channel 338 can include an outer jacket, which can be made of a polymer, Polyurethane, Nylon, PEBAX®, or some other suitable material. Thus, a composite design can be used. The braided wire channel 338 and port channel 318 can be coaxial and can together form a single extended guide channel.

In some embodiments, the access port 300 can be anchored to an airway wall 3 via one or more anchors 309. The anchors 309 can be connected to the distal and/or proximal end of the port body portion 307. In some arrangements, the anchors 309 can be connected directly to the braided wire channel 338. The anchors 309 can include a piercing portion 311 configured to pierce the surrounding airway wall 3. In some embodiments, the anchors can include pad portions 313 that can be configured to limit the depth to which the piercing portions 311 penetrate the airway wall 3 or other body tissue. The anchors 309 can be constructed of a resilient material, such as nitinol, for example but without limitation. In some embodiments, the anchors 309 are constructed of a resilient material such that the anchors 309 can be compressed within a working channel of a deployment device prior to deployment in an airway 2 or other body lumen. In some embodiments, the anchors 309 include articulated arm portions between the piercing members and the anchor attachment points on the port body portion 307. U.S. Pat. Nos. 6,293,951, 6,592,594, 6,722,360, 6,929,637, 7,533,671, 7,691,151, 7,875,048 and U.S. Publication Nos. 2003/0154988, 2003/0181922, 2003/0195385, and 2003/0212412 provide examples of embodiments of anchors 309 and are hereby incorporated by reference herein in their entireties.

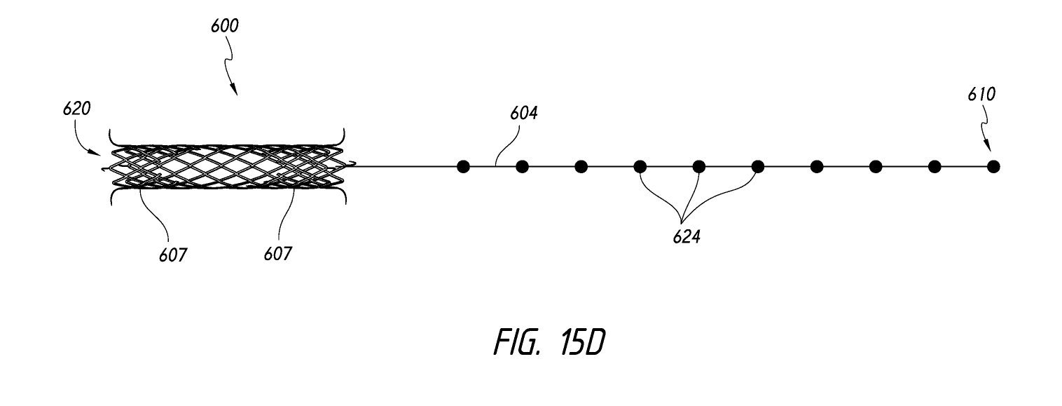

FIG. 13 illustrates an embodiment of an access port 400 that includes a coil portion 432 attached to the proximal end of the port body portion 407. The coil portion 432 can comprise a coil channel 438, which can be coaxial with the port channel 407 and can form an extended channel guide channel together with the port channel 407. The access port 400 can include anchors 409 that can have characteristics similar or identical to the anchors 309 described above.

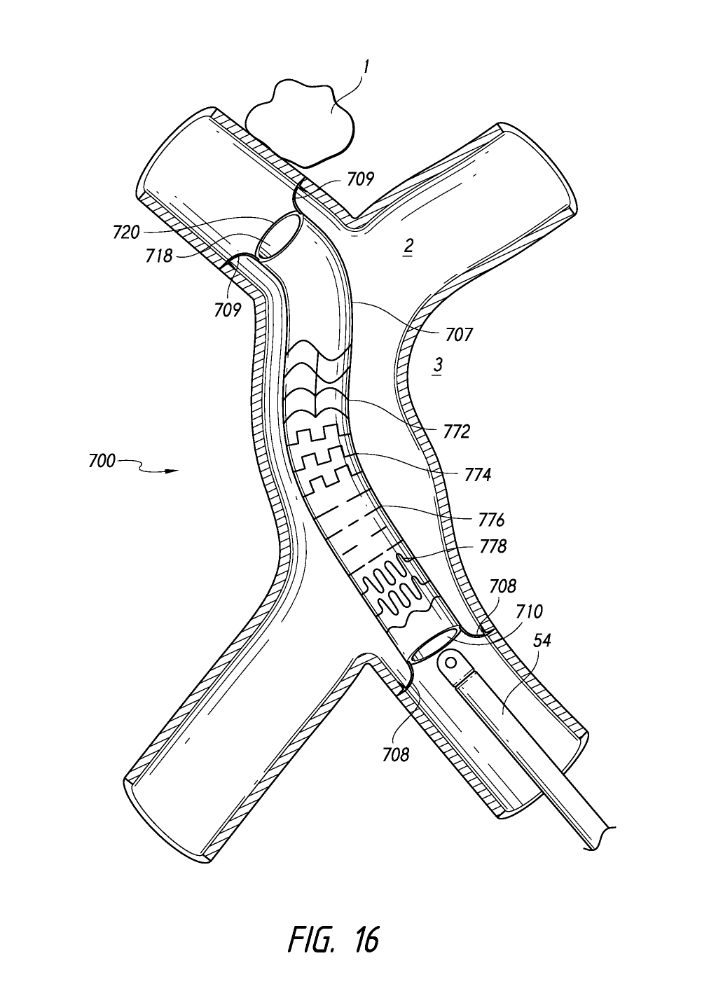

FIG. 14 illustrates an embodiment of an access port 500. The access port 500 can include a body portion 507. In some embodiments, the access port 500 includes a removal rod 554 extending from the proximal end of the body portion 507. The removal rod 554 can include a removal point 553 on the distal end of the removal rod 554. In some embodiments, the access port 500 includes a coil portion 532 that can be attached to the proximal end of the removal point 553 and can extend in the proximal direction from the access port 500. The coil portion 532 can comprise a coil channel 538 through which instruments, catheters, and/or other medical devices could be guided. The removal point 553 can include a redirection member 518 that can help guide an instrument toward the airway wall 3. In some embodiments, as illustrated, the redirection member 518 comprises a hole drilled through the removal point 553. In some embodiments, the redirection member 518 comprises a spigot for redirecting instruments away from the axis of the removal rod 554. In some embodiments, the redirection member 518 comprises a channel in the removal point 553. In some embodiments, a portion of the proximal end of the removal point 553 slants away from the axis of the removal rod 554 and toward the airway wall 3. The access port 500 can include anchors 509 that can have characteristics similar or identical to any of the anchors 309, 409 described above.

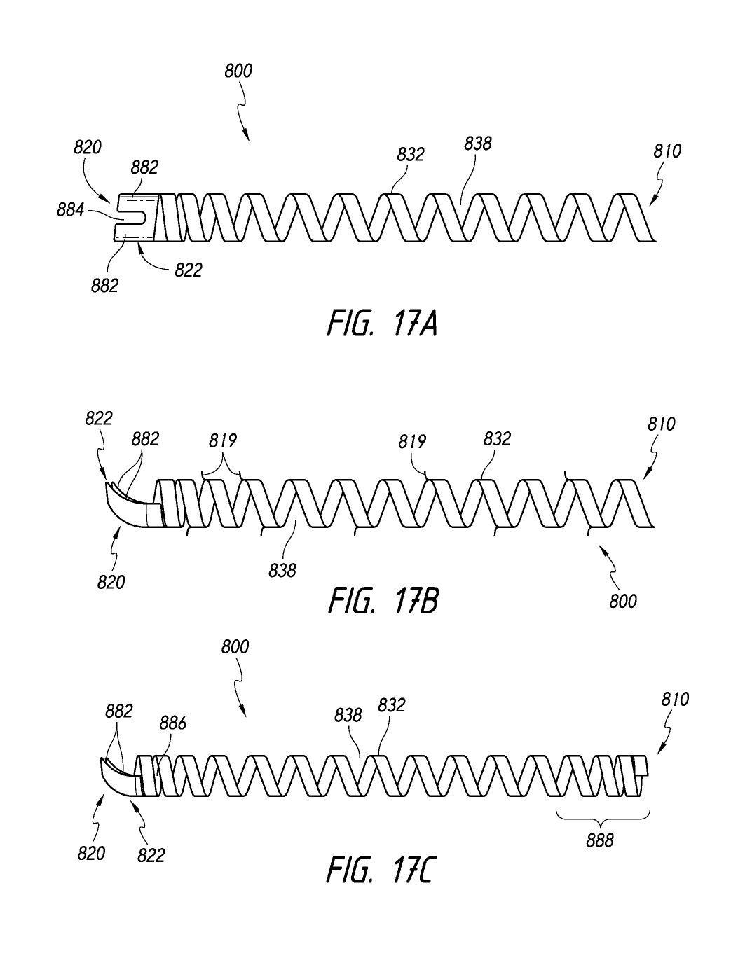

FIGS. 15A-15D illustrate embodiments of an access channel 600. The access channel 600 can include a channel body portion 607. In embodiments, the channel body portion 607 is substantially cylindrical. In some embodiments, portions of or the entire channel body portion 607 are substantially impermeable. In some embodiments, as illustrated in FIG. 15A, the channel body 607 can include a plurality of holes or openings. The channel body portion 607 can have a proximal end and a distal end. In some embodiments, the channel body portion 607 has a polygonal cross-section or a generally cylindrical cross-section.

The channel body portion 607 can include one or more radiopaque portions 627. In some embodiments, the channel body portion 607 includes a side port 629. In configurations with the side port 629, rotational orientation can be somewhat important during implantation. Accordingly, the radiopaque portions 627 can be configured such that the rotational orientation of the channel body portion 607 can be visualized. In some configurations, the channel body portion 607 can comprise landmarks (e.g., dimples or the like) such that the location of the side port 627 can be better visualized.

In some embodiments, the access channel 600 can include one or more anchors 608,609. The one or more anchors can include one or more distal anchors 609 and/or one or more proximal anchors 608. Any suitable anchoring configuration can be used. Desirably, the access channel is secured against substantial movement in both the distal and the proximal directions.

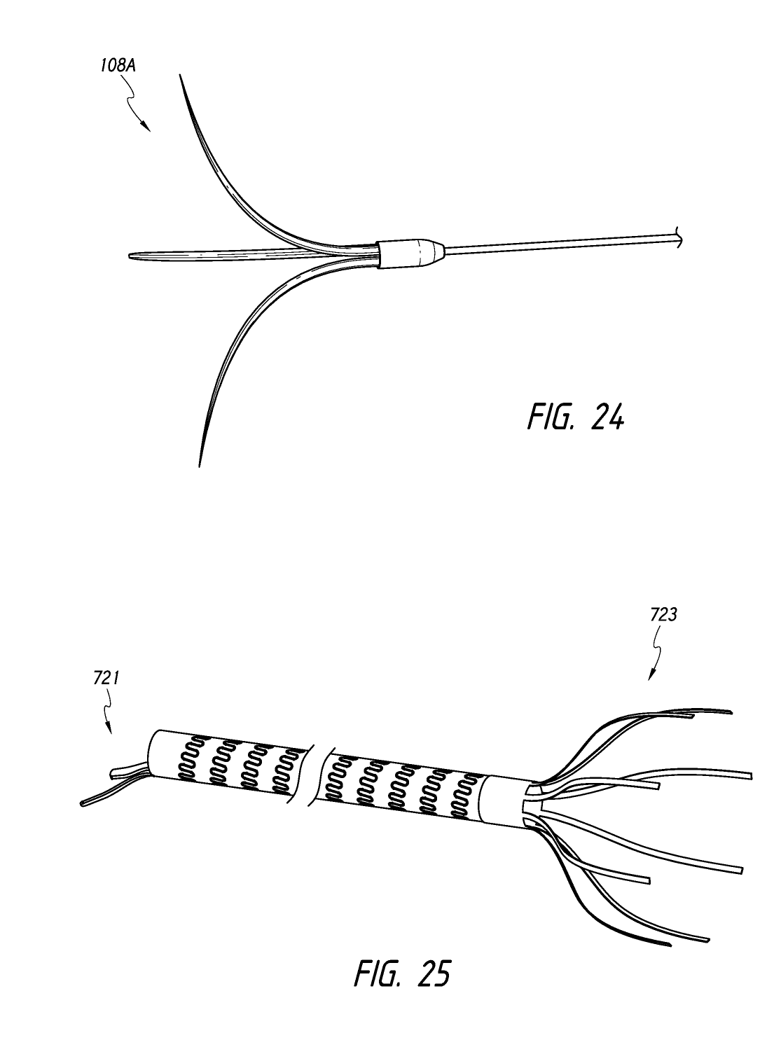

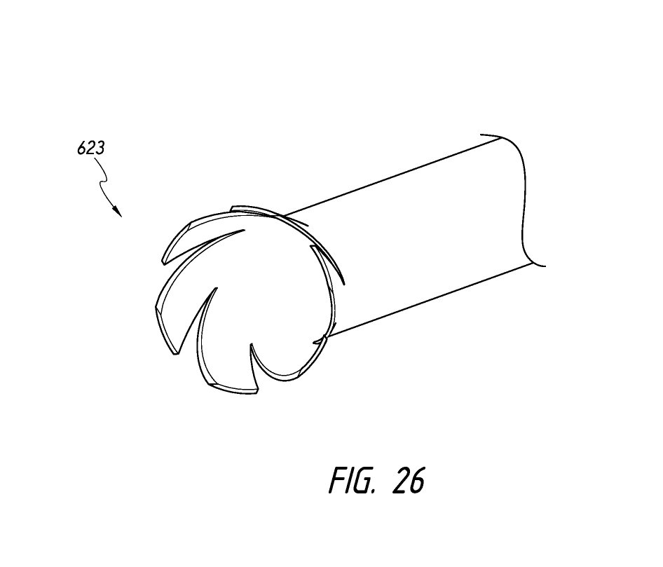

The access channel 600 can include an interior guide channel 618. In some embodiments, the interior guide channel 618 extends from the proximal end of the channel body portion 607 to the distal end of the channel body portion 607. In some embodiments, the guide channel 618 extends from the proximal end of the body portion 607 to the side port 629. In some embodiments, the proximal end of the body portion 607 can include an outwardly flared portion that could facilitate easier insertion of instruments into the proximal end of the guide channel 618. In some embodiments, the proximal end of the body portion 607 can include a plurality of outwardly projecting fingers forming a basket 723, similar to the basket 723 shown in FIG. 25. In some embodiments, the proximal end of the body portion 607 has outwardly projecting strips 623, as illustrated in FIG. 26. In some configurations, the proximal end of the body portion 607 can taper to allow a bronchoscope or the like to dock onto the proximal end of the body portion 607.

FIG. 15B illustrates a deployed configuration of the access channel 600. In some embodiments, the access channel 600 is configured to compress into the working channel of a catheter or other delivery device. As illustrated in FIG. 15B, the access channel 600 can be delivered to the site of a nodule 1 such that the side port 629 aligns with the nodule 1. In some embodiments, the access channel 600 can be deployed in an airway 2 or other body lumen proximal to the nodule 1. In some embodiments, the access channel 600 can be deployed such that the radiopaque portions 627 straddle or otherwise correspond to the location of the nodule 1. In some configurations, the access channel 600 has an outside dimension that is between 110% and 40% of the passage cross-section. In some configurations, the access channel has an outside dimension that is between about 95% and about 70% of the passage cross-section. In some configurations, the access channel 600 is radially expandable (e.g., like a stent) such that the access channel can hold an airway passage open in a region of a nodule.

In some embodiments, the access channel 600 can include a guide wire 604 extending from the proximal end of the body portion 607. The guide wire 604 can have a proximal end 610 with a proximal structure similar to those described above (e.g. the rounded end cap, bristled end, etc.). The guide wire 604 can pass through one or more generations of the bronchial tree, as illustrated in FIG. 15C. In some embodiments, the guide wire 604 can include one or more radiopaque distance markers 624. In some embodiments, the distance markers 624 can provide visual indication of knotting or bending of the guide wire 604. In some embodiments, the markers 624 can provide an indication of the path to the nodule through the bronchial passageways. In some embodiments, the markers 624 can be used by a navigation system to help direct a user to the site of the nodule. In some embodiments, the distal end 620 and/or the proximal end of the body portion 607 can include a film cover to inhibit or reduce the likelihood of bodily tissue, fluid or foreign substances from entering the guide channel 618.

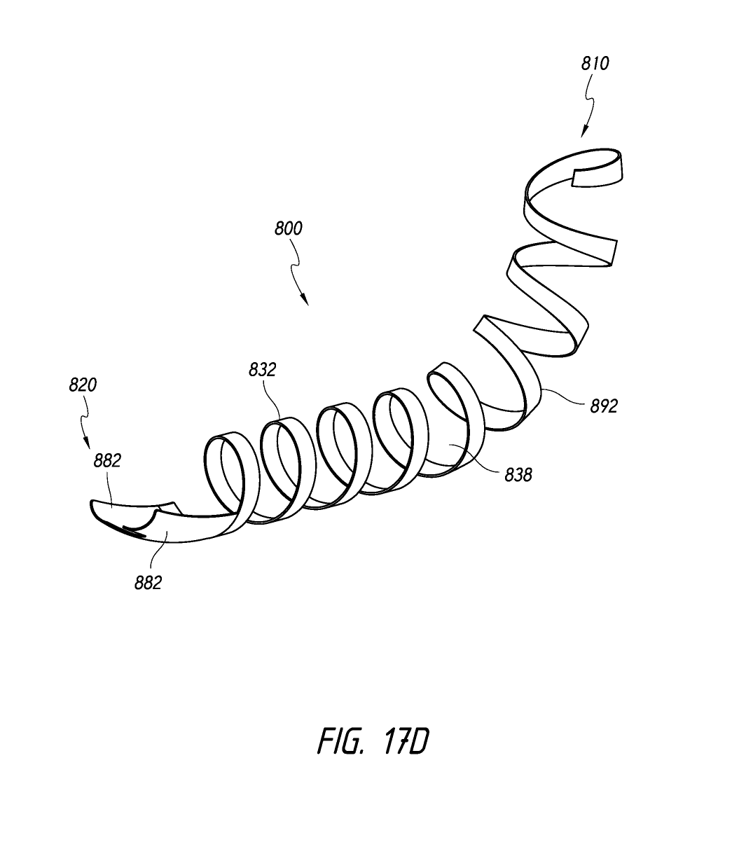

FIG. 16 illustrates an embodiment of an access channel 700 that can, in some embodiments, span one or more generations of the bronchial tree. The access channel 700 can have a proximal end 710 and a distal end 720. In some embodiments, the access channel 700 includes a channel body portion 707. In embodiments, the channel body portion 707 is substantially cylindrical. In some embodiments, the channel body portion 707 has a polygonal cross-section. In some embodiments, the channel body portion 707 can be constructed of a PTFE-fined braid of reinforced PEBAX® or some other suitable material. In some embodiments, the outside of the body portion 707 can be constructed of PEBAX® 72D or some other suitable material. In some embodiments, the number of braid crossings per inch can be varied along the length of the body portion 707, as illustrated in FIGS. 32 and 33. Variation in the number of braid crossings per inch can change the flexibility of the body portion 707 along its length.

The channel body portion 707 can have a proximal end and a distal end. The channel body portion 707 can include an interior guide channel 718. In some embodiments, the interior guide channel 718 extends from the proximal end 710 of the channel body portion 707 to the distal end 720 of the valve body portion. In some embodiments, the diameter of the guide channel 718 is greater than about 1 mm and/or less than about 5 mm. In some embodiments, the diameter of the guide channel 718 is approximately 2 mm. In some embodiments, the length of the channel body portion 707 can be greater than about 4 cm and/or less than about 15 cm. In some embodiments, the channel body portion 707 is greater than 5 cm length and less than 9 cm in length. In some embodiments, the channel body portion 707 is approximately 10 cm in length.

In some embodiments, the access channel 700 can include one or more anchors 708,709. The one or more anchors can include one or more distal anchors 709 and/or one or more proximal anchors 708. In some embodiments, the access channel 700 can include a guide wire extending from the proximal end 710 of the access channel 700. In some embodiments, the proximal end 710 of the channel body portion 707 can include an outwardly-tapered portion to help instruments 54 (e.g. biopsy forceps, cytology brushes, etc.) enter the proximal end 710 of the interior guide channel 718. In some embodiments, the proximal end 710 of the body portion 707 can include a plurality of outwardly projecting fingers forming a basket 723, as shown in FIG. 25. In some embodiments, the proximal end 710 of the body portion 707 has outwardly projecting strips 623, as illustrated in FIG. 26. In some embodiments, the proximal end 710 of the body portion 707 can be configured to “dock” with the distal end of a bronchoscope or other delivery device. For example, the proximal end 710 of the body portion 707 can be sized such that it fits within the working channel of a bronchoscope. In such an embodiment, the proximal end 710 of the body portion 707 could be mated with the distal end of the working channel of a bronchoscope, thus effectively extending the working channel of the bronchoscope further into the periphery of the lung or other body organ or lumen. In some embodiments, the proximal end 710 of the body portion 707 can be sized such that the entire distal end of the bronchoscope or other delivery device would fit inside the proximal end 710 of the body portion 707. In some embodiments, the distal end 720 of the access channel 700 can include a directional member 721, which can help guide an instrument toward a peripheral nodule 1, as illustrated in FIG. 25. For example, the directional member 721 can include one or more projections configured to rest against or into the wall of the passage in which the access channel is implanted. The projections of the directional member 721 can deflect (e.g., bend) the distal end 720 of access channel 700 away from the wall onto or into which the projections are engaged. Such a deflection can help to direct (e.g., point) the distal end 720 of the access channel 700 toward a nodule or other site of interest. The projections of the directional member 721 can be oriented (e.g., bent, widened) prior to, during, or after deployment of the access channel 700 to vary the extent to which the distal end 720 is deflected.

In some embodiments, the body portion 707 of the access channel 700 can be constructed of a stainless steel or nitinol hypotube or some other resilient material. The body portion 707 of the access channel 700 can be cut using a laser, photochemical mill, water jet or other suitable process. In some configurations, the body portion 707, or a segment thereof can be cut in a braided pattern 772. In some configurations, the body portion 707, or a segment thereof, can be cut in a jigsaw pattern 774. In some configurations, the body portion 707, or a segment thereof, can be cut in a stop cut pattern 776. In some configurations, the body portion 707, or a segment thereof, can be cut in a serpentine pattern 778. In some configurations, the body portion, or a segment thereof, can be cut in one or more of the above patterns. In some configurations, the body portion is not cut. Cutting the body portion 707 can increase the flexibility of the access channel 700 and allow the access channel to more easily navigate tortuous airways 2 or other body lumens. In some embodiments, the proximal end 710 of the body portion 707 is cut to have increased flexibility. In some embodiments, cuts in the body portion 707 can be sealed with heat shrink from the interior of the guide channel 718, from the exterior of the body portion 707, or from both sides. In some embodiments, cuts in the body portion 707 can be sealed with heat shrink from the exterior of the body portion 707. In some embodiments, PTFE, PEBAX®, or some other suitable material can be used to coat the interior of the guide channel 718 and/or the exterior of the body portion 707.

In some embodiments, the access channel 600, 700 is deployed at a site of interest (e.g., a nodule) using a bronchoscope of other delivery device (e.g., an endoscope or delivery catheter). The access channel 600, 700 can be stored in a working channel or other lumen of a delivery device before deployment. In some embodiments, the access channel 600, 700 is configured to radially compress into the working channel or other lumen of a delivery device.

The delivery device can navigate to the site of interest using a visualization device, such as, for example, an ultrasound probe. The visualization device can be sized and shaped to fit within the working channel or other lumen in which the access channel 600, 700 is stored prior to deployment. In some embodiments, the visualization device is sized and shaped to fit within (e.g., able to pass through) the access channel 600, 700 when the access channel 600, 700 is contained within the lumen of the delivery device. The access channel 600, 700 and/or lumen can be filled with a gel or other fluid to facilitate measuring continuity of the visualization device (e.g., ultrasound continuity of an ultrasonic probe).

The visualization device can be used to locate the specific location (e.g., the radial and/or circumferential location with respect to the delivery device) of the site of interest (e.g., nodule) near which the access channel 600, 700 is to be deployed. In some embodiments, the visualization device is configured to detect surface and/or structural features (e.g., echoginically unique features) of the access channel 600, 700. Such echogenically unique surface and/or structural features could comprise features that have different echogenicity from the portions of the access channel 600, 700 adjacent to or surrounding the features. For example, as illustrated in FIG. 25, the access channel 600, 700 can include one or more directional members 721. In some such embodiments, the visualization device (e.g., ultrasound probe) can be used to detect the rotational orientation of the one or more direction members 721 (or other features such as, for example, cut patterns, side ports) of the access channel 600, 700. The access channel 600, 700 can be rotated within the lumen of the delivery device to rotationally align the relevant feature (e.g., the direction members 721, cut patterns, side ports 629) to a desired rotational position. For example, the direction member 721 can be aligned on the circumferentially opposite side of the lumen into which the access channel 600, 700 is deployed from the site of interest (e.g., a nodule). In some such variants, the direction member 721 can direct the distal end of the access channel 600, 700 toward the site of interest.

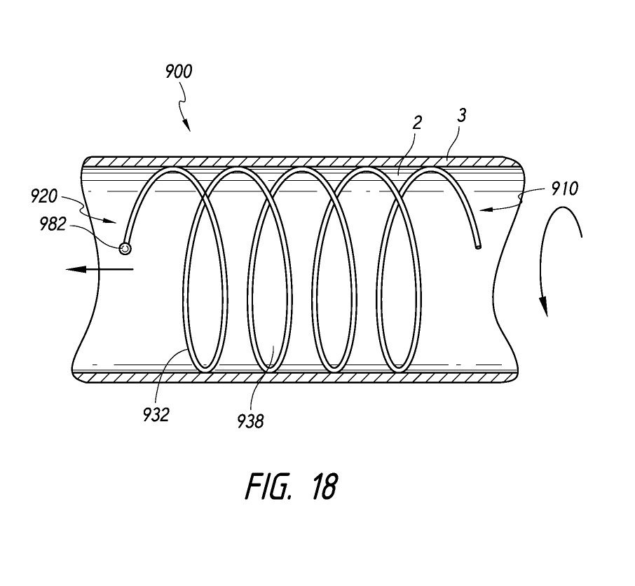

FIGS. 17A-17D illustrate an embodiment of a flat coil access channel 800 that can be used to provide repeatable access to a nodule 1 in the lung or in another body organ or cavity. In some embodiments, the flat coil access channel 800 has a proximal end 810 and a distal end 820. The flat coil access channel 800 can be formed by coiling a flat strip of nitinol or other suitable material. The access channel 800 can include a flat coil portion 832. In some embodiments, the flat coil portion 832 defines a coil channel 838. The coil channel 838 can extend from the proximal end 810 of the access channel 800 to the distal end 820 of the access channel 800.

In some embodiments, the flat coil access channel 800 can include a spigot 822 on the distal end 820 of the flat coil access channel 800. The spigot 822 can have one or more tines 882. In some embodiments, the spigot 822 has a notch 884 cut into it. In some embodiments, the notch 884 can allow the spigot 822 to compress within the working channel of a catheter or other delivery device. The spigot 822 can be configured to direct an instrument toward a nodule 1 when the instrument is inserted through the coil channel 838. The angle of the spigot 822 with respect to the central access of the coil channel 838 can vary depending on the application of the flat coil access channel 800 and/or the relative location of the target nodule 1.

In some configurations, the flat coil access channel 800 can include one or more reduced pitch portions 886,888. The one or more reduced pitch portions 886,888 can form a collar. In some embodiments, a collar on the proximal end 810 of the flat coil access channel 800 can help make it easier to insert instruments into the proximal end 810 of the coil channel 838. In some embodiments, the flat coil access channel 800 can include one or more anchors 819. The anchors 819 can help reduce the likelihood that the access channel 800 will rotate or move in a proximal or distal direction within the airway 2 in which the access channel 800 is deployed. In some embodiments, the flat coil access channel 800 can be configured to provide support to the airway 2 in which it is deployed. In some embodiments, the flat coil access channel 800 can help reduce the likelihood that the airway 2 in which the access channel 800 is deployed will collapse. In some embodiments, a flat coil access channel 800 can have one or more pre-set bends 892, as illustrated in FIG. 17D. Pre-set bends 892 can help the flat coil access channel 800 conform to tortuous airways 2.

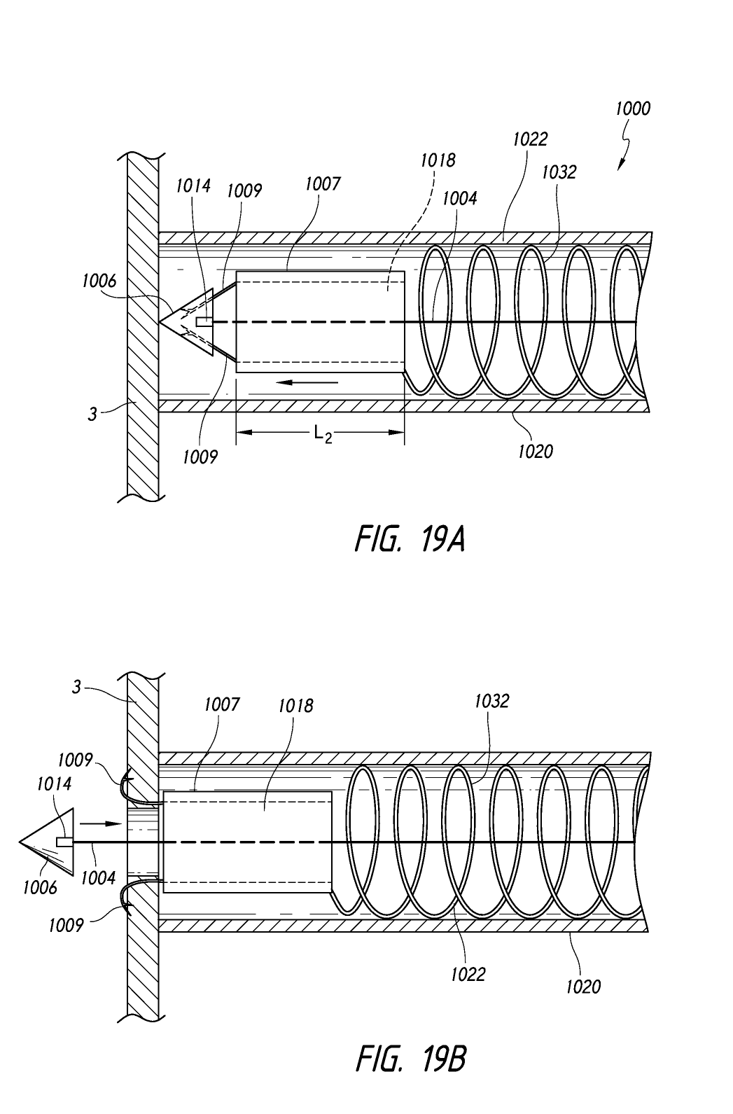

FIG. 18 illustrates an embodiment of a coil tunneling device 900 that can be used to provide repeatable access to a nodule 1 in a lung or other body organ or lumen. The coil tunneling device 900 can have a proximal end 910 and a distal end 920. The coil tunneling device 900 can include a coil portion 932. The coil portion 932 can define a coil channel 938 that can extend from the proximal end 910 of the tunneling device 900 to the distal end 920 of the tunneling device 900 and through which instruments can be navigated. The distal end 920 of the tunneling device can include a distal point 982. In some embodiments, the distal point 982 could include a marker (e.g. a radiopaque marker) to assist in visualization of the tunneling device 900 as it is positioned within the body.

In some embodiments, the tunneling device 900 can be navigated through airways 2 or other body lumens by rotating the tunneling device 900 as illustrated in FIG. 18. As the tunneling device 900 is turned in the direction shown in FIG. 18, the spiral pattern of the coil portion 932 will advance the tunneling device 900 in the distal direction. In some configurations, the tunneling device 900 can be moved in the proximal direction by turning the tunneling device 900 in the direction opposite that shown in FIG. 18. In some embodiments, the tunneling device 900 can be steered using one or more guide wires.

In some configurations, the inner diameter of the coil channel 932 can be increased by fixing the distal end 920 of the coil portion 932 in place while unwinding the coil portion 932 from the proximal end 910. This technique could be used to increase the diameter of an airway 2 and could provide easier access to portions of the lung distal to the tunneling device 900.

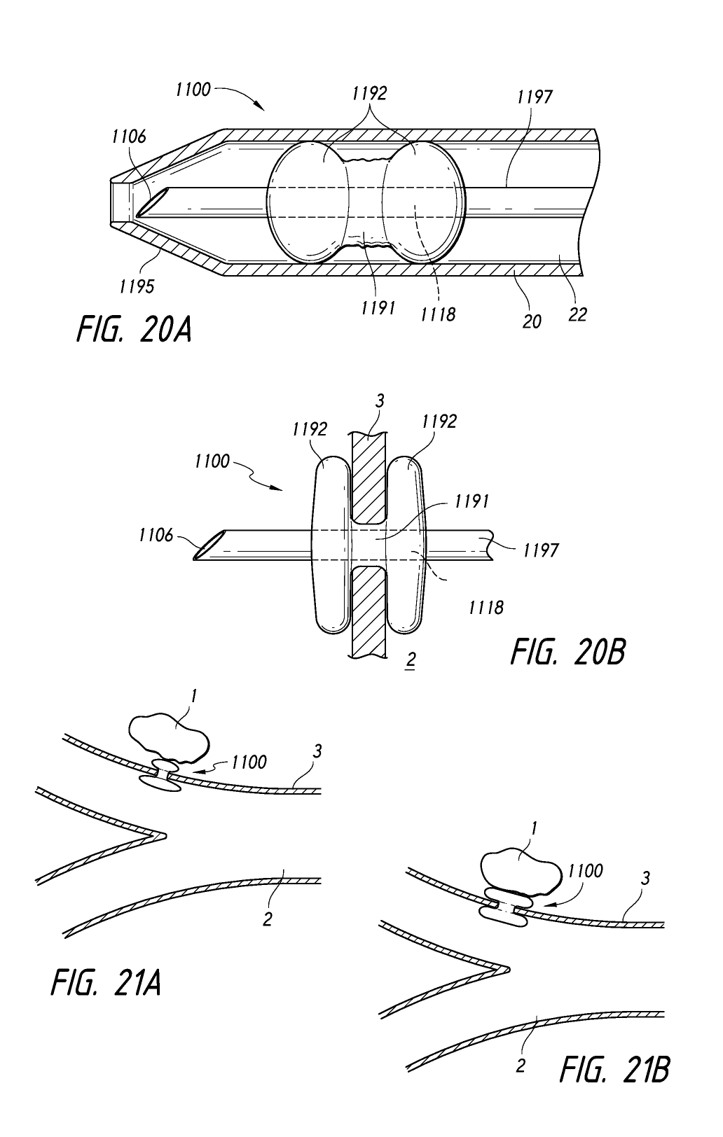

FIGS. 19A and 19B illustrate deployment apparatus 1000 for delivering a port access device to a body lumen. The deployment apparatus 1000 can include an outer sheath 1020 defining a working channel 1022. The port access device can have a body portion 1007 defining a port channel 1018. In some embodiments, the length L2 of the body portion can be greater than about 2 cm and/or less than about 20 cm. In some embodiments, the length L2 of the body portion 1007 is approximately 10 cm. Many variations are possible. In some embodiments, the port access device can be configured to be compressed within the working channel 1022 of the deployment apparatus 1000. In some embodiments, the port access device can include one or more anchors 1009. In some embodiments, the deployment apparatus 1000 can further comprise a cone portion 1006 into which the one or more anchors 1009 of the port access device can be compressed prior to deployment of the port access device. In some embodiments, the cone portion 1006 includes a notch 1014. The one or more anchors 1009 can be inserted into a recess defined within the cone portion 1006 by inserting a first anchor 1009 into the notch 1014, turning the cone portion 1006 with respect to the port access device, inserting a second anchor 1009 into the notch 1014, and so on until all of the anchors 1009 are compressed within the cone portion 1006. The cone portion 1006 can include a cone wire 1004 that can be used to move the cone portion 1006 in the distal and or proximal direction.

In some embodiments, the deployment apparatus 1000 can further include a push coil 1032 or other suitable translatable flexible component located in the working channel 1022 proximal to the body portion 1007 of the port access device. The push coil 1032 can be used to push the port access device in the distal direction with respect to the sheath 1020. In some embodiments, the deployment apparatus 1000 can be used in conjunction with a visualization system, including but not limited to any of those visualization or navigation systems discussed above. In some arrangements, the deployment apparatus 1000 can be used in conjunction with fluoroscopy or the like.

A method of deploying the port access device can include navigating the distal end of the deployment apparatus 1000 to an airway wall 3. The push coil 1032 can be used to push the cone portion 1006 through the airway wall 3 or other suitable location along with the anchors 1009. As illustrated in FIG. 19B, the cone portion 1006 can punch a hole through the airway wall 3. The cone portion 1006 of the deployment apparatus 1000 then can be pushed in the distal direction with respect to the port access device to allow for release and deployment of the anchors 1009. After the anchors 1009 are deployed, as shown in FIG. 19B, the cone portion 1006 can be pulled in the proximal direction through the hole in the airway wall 3 and through the port channel 1018 and the deployment apparatus 1000 can be withdrawn from the deployed port access device. Although the embodiment of the delivery apparatus 1000 shown in FIGS. 19A and 19B has been used to deploy a port access device aligned substantially perpendicular to an airway wall, the delivery apparatus could also be used to deploy a port access device within an airway 2 substantially parallel to one or more airway walls 2, as illustrated in FIGS. 12-14, or at any angle there between.

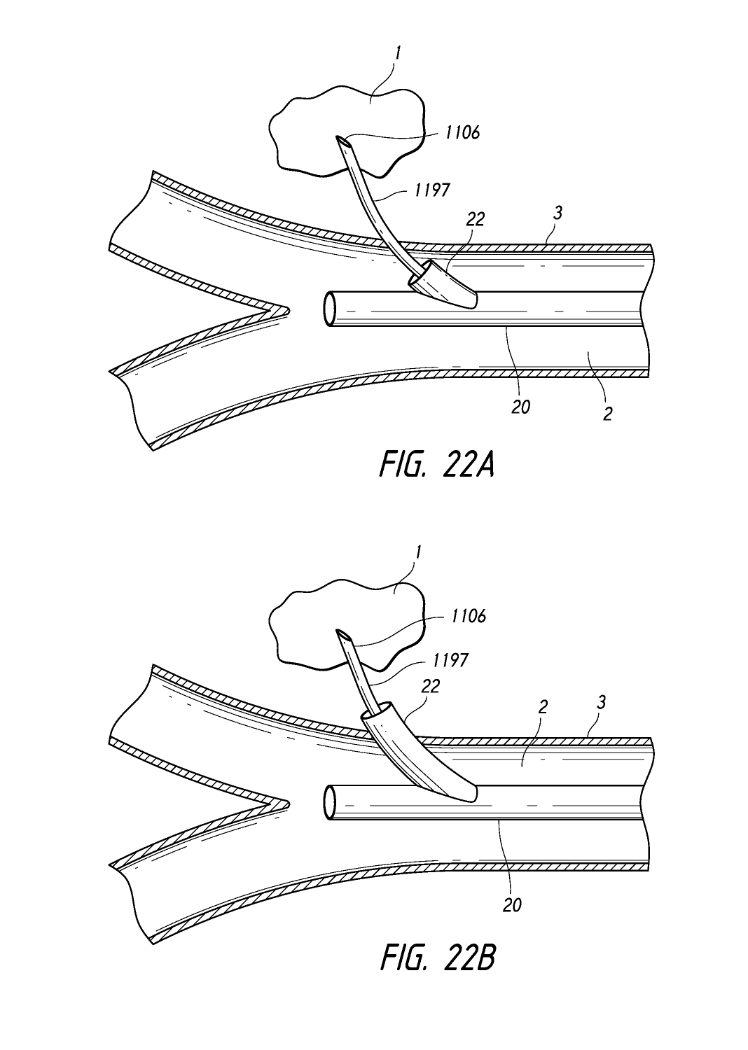

It is sometimes desirable to provide repeatable access through an airway wall 3 into peripheral tissue surrounding an airway 2. This may be the case where a target nodule 1 or other area of interest lies outside of an airway 2 in the surrounding tissue/cavity. One way to accomplish this is to deploy a transluminal access port 1100, as illustrated in FIGS. 20A and 20B. The transluminal access port 1100 can include a channel portion 1191. The transluminal access port 1100 could include one or more expansion portions 1192. In some embodiments, the expansion portions 1192 are constructed of a compressible resilient material that can be compressed within the working channel 22 of a catheter 20 or other delivery device and can expand to an expanded state upon withdrawal or ejection from the working channel 22. The transluminal access port 1100 can include a port channel 1118 extending from the proximal end of the access port 1100 to the distal end of the access port 1100 or some segment therebetween. The channel portion 1191 can be constructed of a material sufficiently rigid to withstand compressive forces from the surrounding airway wall 3 upon deployment of the access port 1100 in an airway wall 3.

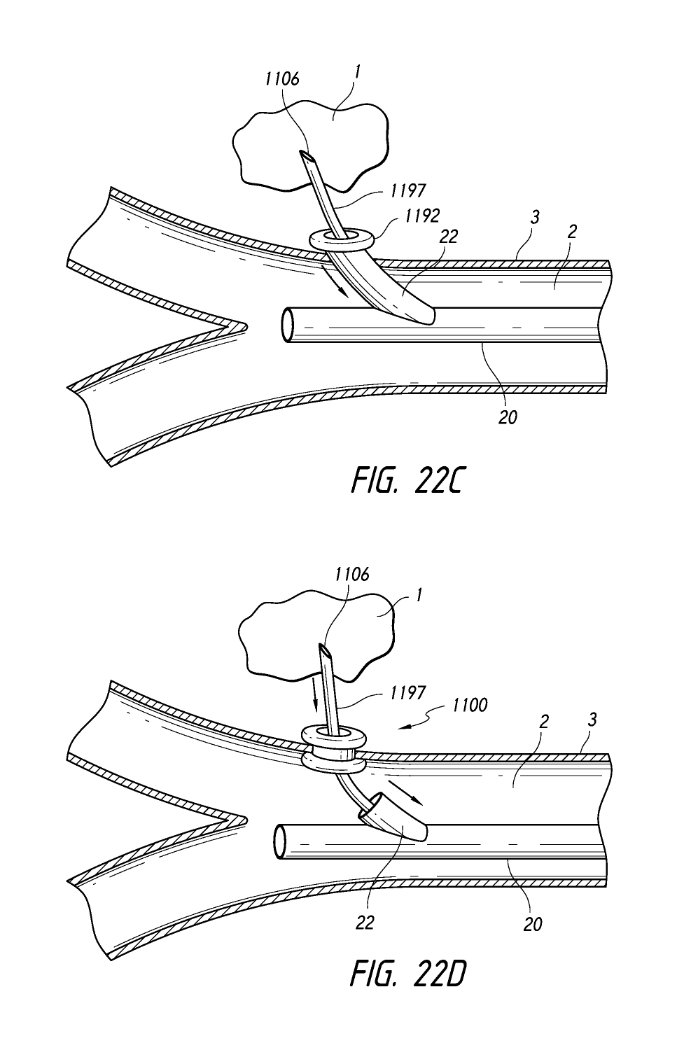

FIG. 20B shows the transluminal access port 1100 in an expanded configuration. As illustrated, the transluminal access port 1100, upon installation in an airway wall 3, can create communication from the interior of the airway 2 to the exterior of the airway wall 3. FIG. 21A shows an installed transluminal access port 1100. As illustrated, the access port 1100 can create direct access from the airway 2 to a location of a nodule 1 outside the airway 2. FIG. 21B illustrates the tendency of the access port 1100 to expand after installation in an airway wall.

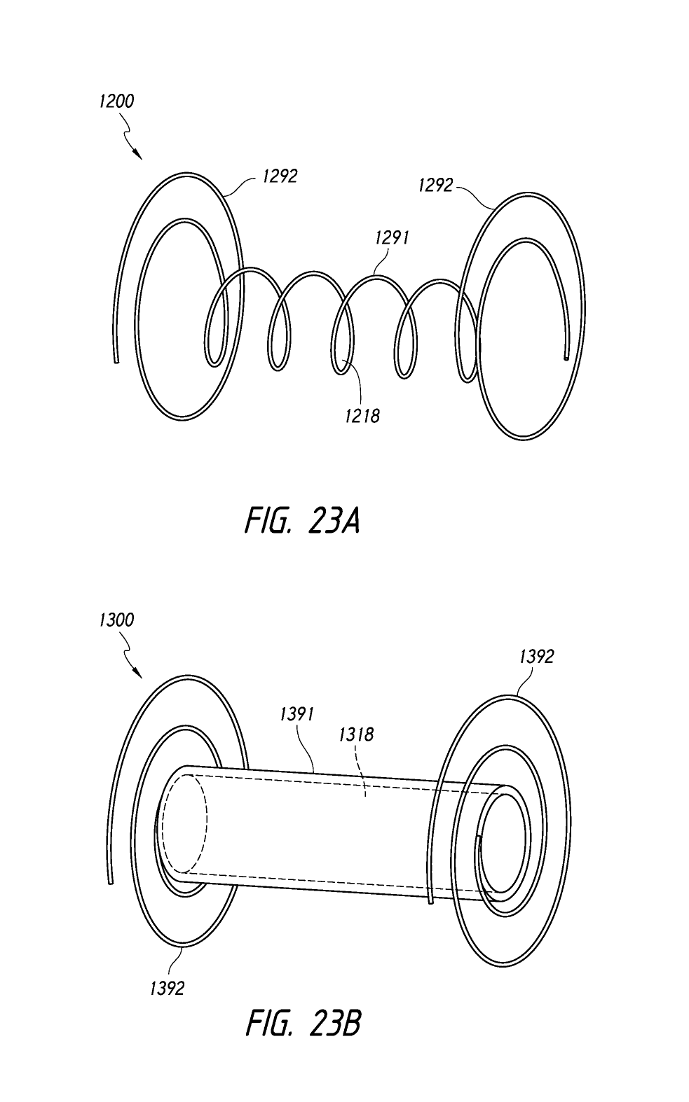

FIGS. 22A-22D illustrate an apparatus and method for installing a transluminal access port 1100 in an airway wall 3. As illustrated, a needle 1197 or other suitable device can be used to penetrate an airway wall 3. In some embodiments, the needle 1197 includes a piercing portion 1106 that can help the needle 1197 to penetrate the airway wall 3. A delivery device 22 of a catheter 20 then can be inserted along with the access port 1100 through the hole created by the needle 1197. In some embodiments, the delivery device 22 includes an inwardly-tapered portion 1195 which can help the delivery device 22 to move through the hole created by the needle 1197.

After the access port 1100 and delivery device 22 are inserted through the airway wall 3, the delivery device 22 can be withdrawn relative to the access port 1100, as illustrated in FIGS. 22C and 22D. As the delivery device 22 is withdrawn from the access port 1100, the distal expansion portion 1192 expands to a diameter greater than the diameter of the hole in the airway wall 3 created by the needle 1197 and the delivery device 22. Upon full withdrawal of the delivery device 22 from the transluminal access port 1100, the proximal expansion portion 1192 of the access port 1100 can expand to a diameter greater than the diameter of the hole in the airway wall 3 created by the needle 1197 and the delivery device 22. After the expansion of the expansion ports 1192, the needle 1197 can be withdrawn through the port channel 1118 of the access port 1100. Once installed, the access port can provide easy and repeatable access to the site of the nodule 1. In some embodiments, the expansion ports 1192 can provide compressive force on the tissue surrounding the channel portion 1191. Such a compression force could help reduce the compressive forces exerted on the channel portion 1191 by the tissue surrounding the channel portion 1191.

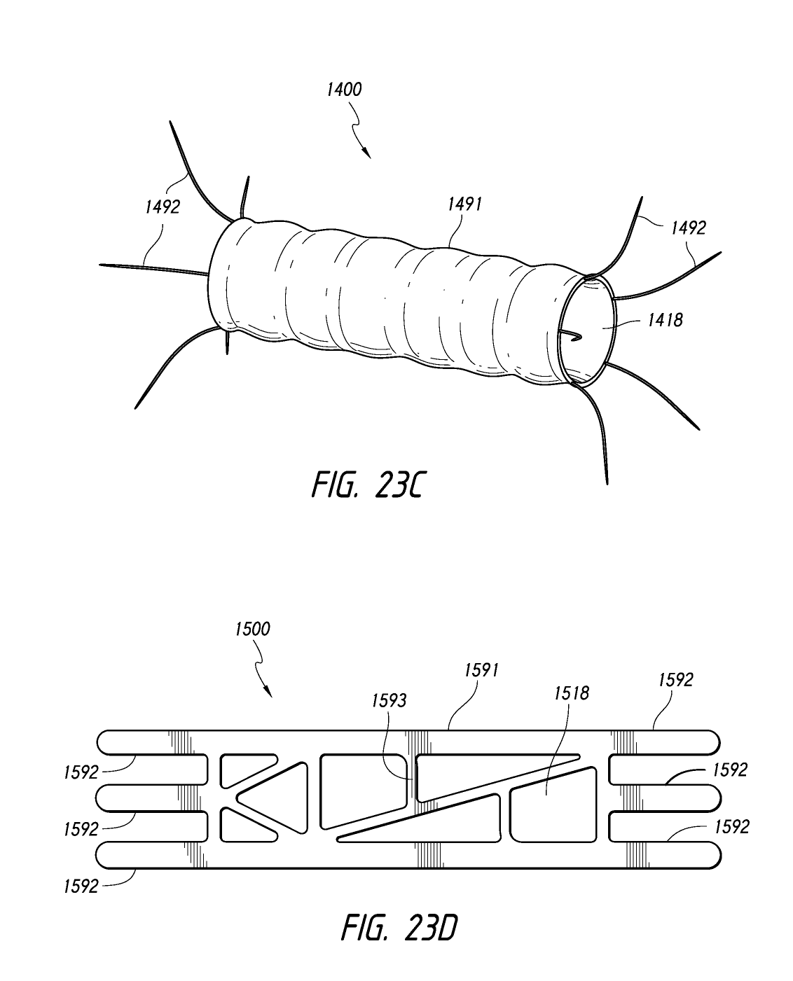

FIGS. 23A-23F illustrate embodiments of transluminal access ports. In some embodiments, a transluminal coil access port 1200 can include a coil channel portion 1291. The coil channel portion 1291 can define a coil port channel 1218 extending from the proximal end of the coil access port 1200 through the distal end of the coil access port 1200. The coil access port 1200 can also include coil expansion portions 1292 on the proximal and distal ends of the coil access port 1200.

In some embodiments, a transluminal access port 1300 can include a channel portion 1391. The channel portion 1391 can define a port channel 1318 extending from the proximal end of the access port 1300 to the distal end of the access port 1300. The transluminal access port 1300 can also include coil expansion portions 1392 on the proximal and distal ends of the coil access port 1300.

In some embodiments, a transluminal anchored access port 1400 can include a channel portion 1491. The channel portion 1491 can define a port channel 1418 extending from the proximal end of the access port 1400 to the distal end of the access port 1400. The transluminal anchored access port 1400 can also include radial anchors 1492 on the proximal and distal ends of the coil access port 1400. The radial anchors 1492 can be configured to expand radially away from the channel portion 1491 and to anchor the access port 1400 to the airway wall 3 from inside and outside of the airway 2.

FIG. 23D illustrates an embodiment of a transluminal laser-cut access port 1500 that can have a channel portion 1591. The access port 1500 could be constructed of nitinol or some other resilient material. The channel portion 1591 can define a port channel 1518 extending from the proximal end of the channel portion 1591 through the distal end of the channel portion 1591. The channel portion 1591 can include a patterned portion 1593 created by laser-cutting, photochemical milling, and/or some other method of cutting. The access port 1500 can include finger members 1592 on the proximal and distal ends of the channel portion 1591. The finger members 1592 can be configured to transition from a compressed state (as illustrated in FIG. 23D) to an expanded state upon withdrawal from the working channel or other delivery device (as illustrated in FIG. 23E). The expanded finger members 1592 can secure the access port 1500 to an airway wall 3 while the port channel 1518 allows repeatable access between the airway 2 and the site of a nodule 1, for example but without limitation, as illustrated in FIG. 23E.

FIG. 23F illustrates an embodiment of a transluminal laser-cut access port 1600. The access port 1600 can comprise a channel portion 1691. The channel portion 1691 can define a port channel 1618 extending from the proximal end of the channel portion 1691 to the distal end of the channel portion 1691. In some embodiments, the channel portion 1691 can be cut using a laser, photochemical milling, and/or some other means or method of cutting. The cut portion 1675 can be cut in a serpentine, braided, jigsaw, and/or some other pattern. In some embodiments, the access port 1600 can include web portions 1692 on the proximal and distal ends of the channel portion 1691. Upon insertion of the access port 1600 into an airway wall 3, the web portions 1692 can fold outward and back toward the channel portion 1691. By folding in this manner, the web portions 1692 can provide compressive force on the airway wall 3 in which the access port 1600 is deployed.

Components of some or all of the devices described herein can be constructed of biocompatible materials in order to facilitate long term and/or permanent deployment of the device within the body. For example, components can be lined with silver of some other antimicrobial lining to reduce the likelihood that biological material will be deposited on or in the device. In some embodiments, components of the devices can be coated with or constructed of bioabsorbable material. In some embodiments, components of the devices can be coated with porous Teflon to encourage tissue in-growth into the device.

Although this invention has been disclosed in the context of certain embodiments and examples, those skilled in the art will understand that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes or embodiments of the disclosed invention. Thus, it is intended that the scope of the present invention herein disclosed should not be limited by the particular disclosed embodiments described above.

What is claimed is:

1. A device for providing access to a nodule, lesion, or pathological area in a lung or other body organ or lumen, the device comprising:
a channel portion, the channel portion having a proximal end and a distal end, wherein the channel portion defines a lumen, the lumen extending from the proximal end of the channel portion to the distal end of the channel portion; and
at least one anchor member, the anchor member configured to inhibit rotational, proximal, and distal motion of the channel portion with respect to the nodule upon deployment of the device in a body lumen.

2. The device of claim 1, wherein the channel potion further comprises at least one radiopaque marker.

3. The device of claim 1, wherein the device further comprises a guide member.

4. The device of claim 3, wherein the guide member comprises a guide wire.

5. The device of claim 3, wherein the guide member comprises a guide tube.

6. The device of claim 3, wherein the guide member comprises at least one radiopaque marker.

7. The device of claim 1, wherein the channel portion further comprises one or more cut portion, the one or more cut portions configured to increase the flexibility of the channel portion.

8. The device of claim 7, wherein channel portion further comprises a heat shrink.

9. The device of claim 1, wherein the channel portion is further configured to deploy transluminally in the wall of an airway or other body lumen.

10. The device of claim 1, wherein the channel portion is further configured to transition between a compressed state within a working channel of a catheter or other delivery device and an expanded state upon deployment in an airway or other body lumen.

11. The device of claim 1, wherein the channel portion further comprises a port between the proximal end of the channel portion and the distal end of the channel portion.

12. The device of claim 1, wherein the at least one anchor member comprises a piercing portion configured to pierce tissue at or near the nodule, and wherein the at least one anchor member comprises a pad portion configured to limit a depth to which the piercing portion pierces the tissue.

13. The device of claim 1, wherein the channel portion comprises a first anchor coupled with the proximal end of the channel portion and extending proximally from the proximal end of the channel portion when the device is deployed.

14. The device of claim 1, wherein the channel portion comprises a first anchor coupled with the distal end of the channel portion and extending distally from the distal end of the channel portion when the device is deployed.

15. The device of claim 1, wherein the device further comprises a directional member coupled with the distal end of the channel member, the directional member configured to direct the distal end of the channel member toward a wall of an airway or other body lumen upon deployment of the device in an airway or other body lumen.

16. The device of claim 15, wherein the distal end of the channel member has an echogenically unique portion configured to identify the orientation of the directional member.

17. The device of claim 16, wherein the channel member can be rotated prior to deployment to rotationally align the echogenically unique portion with respect to the nodule.

18. The device of claim 15, wherein the channel member can be rotated prior to deployment to rotationally align the directional member with respect to the nodule.

19. The device of claim 15, wherein the anchor member engages the lumen wall upon deployment of the device, engagement of the anchor member with the lumen wall fixing the rotational alignment of the directional member with the nodule.

20. The device of claim 15, wherein the directional member comprises one or more projections.

21. A method of deploying and using a fiducial device for repeatable access to a nodule in a lung or other body organ, the method comprising:
locating a target nodule in the body;
compressing the fiducial device within the working channel of a catheter or other delivery device, the fiducial device comprising a fixation portion and a guide portion;
navigating the catheter or other delivery device to the site of the target nodule;
removing the fiducial device from the working channel of the catheter or other delivery device;
attaching the delivery device to tissue proximate the target nodule;
removing the catheter or other delivery device from the site of the target nodule;
engaging a second catheter or other delivery device with the guide portion of the fiducial device, the second catheter of other delivery device including a treatment or diagnosis instrument;
navigating the second catheter or other delivery device along the guide portion of the fiducial device to the site of the target nodule;
treating or collecting a sample from the target nodule; and
withdrawing the second catheter or other delivery device along the guide portion of the fiducial device.

22. A device for providing access to a nodule in a lung or other body organ or lumen, the device comprising:
a fixation portion having a proximal end and a distal end, the fixation portion comprising one or more anchor portions, the fixation portion configured to be attached to tissue in substantially constant proximity to the nodule; and
a guide portion having a proximal end and a distal end, the guide portion configured to removably engage with a medical instrument, the guide portion further configured to guide the navigation of the medical instrument to the fixation portion.

23. The device of claim 22, wherein the one or more anchor portions are configured to removably attach to tissue.

24. The device of claim 22, wherein the fixation portion is attached directly to the nodule.

25. The device of claim 22, wherein the fixation portion is attached proximal to the nodule.

26. The device of claim 22, wherein the guide portion comprises a guide wire.

27. The device of claim 22, wherein the guide portion comprises a guide channel.

28. The device of claim 22, wherein the guide portion further comprises one or more anchors on the proximal end of the guide portion.

29. The device of claim 22, wherein the fixation portion further comprises one or more radiopaque markers.

30. The device of claim 22, wherein the guide portion further comprises one or more radiopaque markers.

31. The device of claim 22, wherein the device is configured to transition between a compressed state within a working channel of a catheter or other delivery device and an expanded state within an airway or other body lumen.

32. The device of claim 22, wherein the fixation portion is configured to attach to an airway wall.
